(19) United States
(12) Patent Application Publication
(10) Pub. No.: US 2003/0138865 A1
(43) Pub. Date: Jul. 24, 2003

(54) Nucleic acids and polypeptides useful in the diagnosis and treatment of prostate cancer

(75) Inventors: Kyle J. MacBeth, Boston, MA (US); Andrew W. Shyjan, Nahant, MA (US)

Correspondence Address: FISH & RICHARDSON PC, 225 FRANKLIN ST, BOSTON, MA, 02110, US

(73) Assignee: Millennium Pharmaceuticals, Inc., a Delaware corporation

(21) Appl. No.: 10/334,038

(22) Filed: Dec. 30, 2002

Related U.S. Application Data
(63) Continuation of application No. 09/408,628, filed on Sep. 30, 1999.
Application No. 09/408,628 is a continuation-in-part of application No. 09/164,159, filed on Sep. 30, 1998.
Application No. 09/408,628 is a continuation-in-part of application No. 09/163,759, filed on Sep. 30, 1998.

(51) Int. Cl. G01N 33/574

(52) U.S. Cl. 435/7.23

(57) ABSTRACT

The invention is based in part on the discovery of a number of genes (HRPCa 2, HPRCa 3, HRPCa 6/7, HRPCa 8, HRPCa 9, HRPCa 10, HRPCa 13, HRPCa 14, HRPCa 15, and HRPCa 19) which are androgen-inducible in androgen-dependent prostate cancer (e.g., LNCaP cells) cells and constitutively expressed in androgen-independent prostate cancer cells (e.g., LN3 LNCaP cells). Because androgen is required for growth and survival of androgen-dependent prostate cancer cells, HRPCa 2, HPRCa 3, HRPCa 6/7, HRPCa 8, HRPCa 9, HRPCa 10, HRPCa 13, HRPCa 14, HRPCa 15, and HRPCa 19 are therapeutic targets. An agent which decreases the expression or activity of HRPCa 2, HPRCa 3, HRPCa 6/7, HRPCa 8, HRPCa 9, HRPCa 10, HRPCa 13, HRPCa 14, HRPCa 15, or HRPCa 19 may slow or arrest the growth of prostate cancer cells or may kill prostate cancer cells. Moreover, because the genes of the invention are constitutively expressed by androgen-independent prostate cancer cells, they can be used to identify agents useful for the treatment of androgen-dependent and androgen-independent prostate cancer.

### RELATED APPLICATION INFORMATION

This application is a continuation-in-part of U.S. application Ser. No. 09/164,159, filed Sep.30, 1998.

### BACKGROUND OF THE INVENTION

Prostate cancer is the most commonly diagnosed cancer and the second most common cause of death from cancer in American men. Prostate cancer cells often initially rely on androgen (e.g., testosterone) for their growth and maintenance. Therefore, androgen withdrawal, by castration or through the use of an anti-androgenic drug, is a common treatment for prostate cancer. In many cases, however, prostate cancer patients develop androgen-independent prostate cancer so that androgen withdrawal treatment is no longer effective.

The complex process of prostate tumor growth and development involves multiple gene products. Therefore, it is important to identify genes involved in tumor development, growth, and androgen dependence, particularly those genes and gene products that can serve as targets for the diagnosis, prevention, and treatment of prostate cancer.

### SUMMARY OF THE INVENTION

The invention is based in part on the discovery of a number of genes which are androgen-inducible in androgen-dependent prostate cancer cells (e.g., LNCaP cells) and constitutively expressed in androgen-independent prostate cancer cells (e.g., LN3 LNCaP cells). These genes are referred to herein by their clone names: HRPCa 2, HPRCa 3, HRPCa 6/7, HRPCa 8, HRPCa 9, HRPCa 10, HRPCa 13, HRPCa 14, HRPCa 15, and HRPCa 19. These genes are collectively referred to as “the genes of the inventions.” The proteins encoded by the genes of the invention and fragments thereof are collectively referred to as the “proteins of the invention” or “the polypeptides of the invention.” Nucleic acid molecules encoding a protein of the invention or a polypeptide of the invention are collectively referred to as “nucleic acids of the invention.”

HRPCa 2, HRPCa 3, HRPCa 6/7, HRPCa 8, HRPCa 9, HRPCa 10, HRPCa 13, HRPCa 14, HRPCa 15, and HRPCa 19

The polypeptides, and nucleic acids of the invention are potential targets for the development of therapeutic compounds. Because androgen is required for growth and survival of androgen-dependent prostate cancer cells, genes such HRPCa 2, HPRCa 3, HRPCa 6/7, HRPCa 8, HRPCa 9, HRPCa 10, HRPCa 13, HRPCa 14, HRPCa 15, and HRPCa 19 whose expression is increased in the presence of testosterone and constitutively expressed in androgen-independent cancer cells are potential therapeutic targets. An agent which decreases the expression or activity of HRPCa 2, HPRCa 3, HRPCa 6/7, HRPCa 8, HRPCa 9, HRPCa 10, HRPCa 13, HRPCa 14, HRPCa 15, or HRPCa 19 may slow or arrest the growth of prostate cancer cells or may kill prostate cancer cells. Moreover, because the genes of the invention are constitutively expressed by androgen-independent prostate cancer cells, they can be used to identify agents useful for the treatment of androgen-dependent and androgen-independent prostate cancer. For example, an agent which reduces the expression or activity of a gene of the invention, e.g., HRPCa 9, may reduce the growth of androgen-independent prostate cancer or cause an androgen-dependent prostate cancer to become androgen-dependent so that it can be treated with standard androgen withdrawal therapy. Of course, such an agent might also be useful for the treatment of an androgen-dependent prostate cancer.

The genes, polypeptides, and nucleic acids of the invention also have diagnostic uses. For example, by examining the expression of one or more of the genes of the invention (e.g., measuring alterations in the level of a protein or nucleic acid of the invention) in a sample of prostate cancer cells, it may be possible to determine whether the cancer cells are androgen-dependent or androgen-independent. For example, if expression of HRPCa 9 in a sample of prostate cancer cells obtained from a patient is not androgen inducible, i.e., it is constitutively expressed, the prostate cancer is likely androgen-independent. This analysis allows one to predict whether androgen withdrawal therapy is likely to be beneficial to that patient. Thus, the analysis allows one to predict whether a selected compound, e.g., an anti-androgenic compound, can be used to treat the prostate cancer. Importantly, this determination can be made on a patient by patient basis. Thus, one can determine whether or not a particular prostate cancer treatment is likely to benefit a particular patient.

The invention also features diagnostic methods and prognostic methods which can be used to identify patients having or at risk for developing and androgen-independent prostate cancer. The genes, polypeptides, and nucleic acids of the invention can be used to identify cells exhibiting or predisposed to developing prostate cancer (e.g., androgen-independent prostate cancer) thereby diagnosing individuals having, or at high risk for developing, prostate cancer (e.g., androgen-independent prostate cancer).

In the various methods of the invention, gene expression can be measured at the mRNA or protein level. Alternatively, expression can be measured indirectly by measuring the activity of the protein encoded by the identified gene.

In another aspect, the present invention provides a method for detecting the presence of activity or expression of HRPCa 2, HPRCa 3, HRPCa 6/7, HRPCa 8, HRPCa 9, HRPCa 10, HRPCa 13, HRPCa 14, HRPCa 15, or HRPCa 19 in a biological sample by contacting the biological sample with an agent capable of detecting an indicator of HRPCa 2, HPRCa 3, HRPCa 6/7, HRPCa 8, HRPCa 9, HRPCa 10, HRPCa 13, HRPCa 14, HRPCa 15, or HRPCa 19 activity such that the presence of HRPCa 2, HPRCa 3, HRPCa 6/7, HRPCa 8, HRPCa 9, HRPCa 10, HRPCa 13, HRPCa 14, HRPCa 15, or HRPCa 19 activity is detected in the biological sample.

In another aspect, the invention provides a method for treating prostate cancer by modulating the expression or activity of HRPCa 2, HPRCa 3, HRPCa 6/7, HRPCa 8, HRPCa 9, HRPCa 10, HRPCa 13, HRPCa 14, HRPCa 15, or HRPCa 19, the method comprising contacting a cell with an agent that modulates (inhibits or stimulates) HRPCa 2, HPRCa 3, HRPCa 6/7, HRPCa 8, HRPCa 9, HRPCa 10, HRPCa 13, HRPCa 14, HRPCa 15, or HRPCa 19 activity or expression such that HRPCa 2, HPRCa 3, HRPCa 6/7, HRPCa 8, HRPCa 9, HRPCa 10, HRPCa 13, HRPCa 14, HRPCa 15, or HRPCa 19 activity or expression in the cell is modulated. In one embodiment, the agent is an antibody that specifically binds to a polypeptide or protein of the invention.

In another embodiment, the agent modulates expression of HRPCa 2, HPRCa 3, HRPCa 6/7, HRPCa 8, HRPCa 9, HRPCa 10, HRPCa 13, HRPCa 14, HRPCa 15, or HRPCa 19 by modulating transcription of a gene of the invention, modulating splicing of an mRNA produced by a gene of the invention, or modulating translation of an mRNA produced by a gene of the invention. In yet another embodiment, the agent is a nucleic acid molecule having a nucleotide sequence that is antisense to the coding strand of HRPCa 2, HPRCa 3, HRPCa 6/7, HRPCa 8, HRPCa 9, HRPCa 10, HRPCa 13, HRPCa 14, HRPCa 15, or HRPCa 19.

In one embodiment, the methods of the present invention are used to treat a subject having a prostate cancer characterized by aberrant HRPCa 2, HPRCa 3, HRPCa 6/7, HRPCa 8, HRPCa 9, HRPCa 10, HRPCa 13, HRPCa 14, HRPCa 15, or HRPCa 19 protein activity or nucleic acid expression by administering an agent which is a HRPCa 2, HPRCa 3, HRPCa 6/7, HRPCa 8, HRPCa 9, HRPCa 10, HRPCa 13, HRPCa 14, HRPCa 15, or HRPCa 19 modulator to the subject. The modulator can be a peptide, peptidomimetic, or small molecule, e.g., an organic molecule.

The present invention also provides a diagnostic assay for identifying whether a patient has or is at risk of developing prostate cancer, e.g., an androgen-independent prostate cancer, by detecting the presence or absence of a genetic lesion or mutation characterized by at least one of: (i) aberrant modification or mutation of a gene of the invention; (ii) mis-regulation of a gene of the invention; and (iii) aberrant post-translational modification of a protein encoded by a gene of the invention.

In another aspect, the invention provides a method for identifying a compound for the treatment of prostate cancer, e.g., an androgen-independent prostate cancer by identifying a compound that binds to or modulates the activity of a protein of the invention. In general, such methods entail measuring a biological activity of a protein of the invention in the presence and absence of a test compound and identifying those compounds which alter the activity of a protein of the invention.

The invention also features methods for identifying a compound which modulates the expression of a gene of the invention (at the mRNA or protein level) by measuring the expression of a nucleic acid or protein of the invention in the presence and absence of a compound.

HRPCa 13 and Peripheral-Type Benzodiazepine Receptor

HRPCa 13 encodes endozepine, a protein that is involved in steroid synthesis (Hall et al. (1995) Molecular Neurobiol. 10:1-17). Endozepine binds to the peripheral-type benzodiazepine receptor (PBR), a receptor that was first identified as a binding site for benzodiazepine diazepam (valium). PBR is a 60 amino acid protein (GenBank Accession No. U12421). PBR is expressed by nearly all tissues, and is expressed at the highest levels in steroid-producing tissues. In these tissues, PBR is known to regulate the transport of cholesterol from the outer membrane of mitochondria to the inner membrane. This transport step is likely the rate limiting step in steroid biosynthesis. PBR has been cloned (Riond et al. (1991) Eur. J. Biochem. 11:471-480).

Given that HRPCa 13 (endozepine) is a target for the development of therapeutic compounds useful for, e.g., the treatment of cancer, particularly prostate cancer, PBR is also a target for the development of such therapeutic compounds. Thus, the invention features methods for identifying compounds which bind to PBR, reduce the activity of PBR, or reduce the expression of PBR.

The invention features methods for identifying therapeutic compounds by measuring the binding of a test compound to PBR. Compounds which bind to PBR may, like compounds which reduce HRPCa 13 expression or activity may be useful for the treatment of androgen-independent prostate cancer or androgen-dependent prostate cancer. The binding of a test compound to PBR can be measured in the presence or absence of endozepine. Particularly useful compounds are those which successfully compete with, e.g., have a higher affinity for PBR, endozopine.

The invention also features a method for identifying therapeutic compounds by identifying a compound which alters, e.g., decreases, the activity, e.g., an endozopine-induced activity, of PBR.

There are a number of assays which have been used to measure the binding of various ligands (e.g., PK 11195, Ro5-4864, diazepam, (−) PK 14067, and (+) PK 14068) to PBR (Chang et al. (1992) DNA Cell Biol.; Broaddus et al. (1990) Brain Res. 518:199-208; Alexander et al. (1992) Biochem. Pharmacol. 44:269-274; and Olson et al., (1998) Cancer Res. 48:5837-41). These assays can be adapted to identify compounds which reduce the binding of endozapine to PBR or to identify compounds that bind to PBR.

In addition, the invention features a method for identifying a therapeutic compound, e.g., a compound useful for the treatment of androgen-dependent or androgen-independent cancer, e.g., prostate cancer, by measuring the expression of PBR in the presence and absence of a test compound. Compounds, that decrease the expression of PBR, like compounds that decrease the expression of HPRCa 13, may be useful for the treatment of prostate cancer.

PBR may also have diagnostic applications. Increased expression of PBR in prostate cancer cells might be associated with androgen-independent prostate cancer. Thus, by examining the expression of PBR (e.g., by measuring PBR protein or nucleic acid) in sample comprising prostate cells, it may be possible to determine whether the prostate cancer cells are androgen-dependent or androgen independent.

In addition, the invention features diagnostic and prognastic methods which can be used to identify patients having or at risk for developing androgen-independent prostate cancer by measuring the expression or activity of PBR.

The invention also features methods of treating prostate cancer by modulating the expression or activity of PBR, the method comprising contacting a cell with an agent that modulates (inhibits) PBR expression or activity such that PBR expression or activity in the cell is modulated (inhibited or reduced).

Definitions

Differential expression refers to both quantitative, as well as qualitative, differences in the expression pattern of a gene in tumor cells treated with a particular drug and untreated tumor cells. A differentially expressed gene can represent fingerprint gene and/or a target gene.

A fingerprint gene is a differentially expressed gene whose expression pattern can be utilized as part of a prognostic or diagnostic marker for the evaluation of prostate cancer (e.g., androgen independent prostate cancer) or which, alternatively, can be used in methods for identifying compounds useful for the treatment of prostate cancer or evaluating the effectiveness of a prostate cancer treatment. For example, the effect of the compound on the fingerprint gene expression pattern normally displayed in connection with prostate cancer can be used to evaluate the efficacy of the compound as a treatment for prostate cancer or can, additionally, be used to monitor patients undergoing clinical evaluation for the treatment of prostate cancer.

A fingerprint pattern is the pattern generated when the expression pattern of a series (which can range from two up to all the fingerprint genes which exist for a given state) of fingerprint genes is determined. A fingerprint pattern can be used in the same diagnostic, prognostic and compound identification methods as the expression of a single fingerprint gene.

A target gene is a differentially expressed gene involved in prostate cancer such that modulation of the level of target gene expression or of target gene product activity can act to prevent and/or ameliorate symptoms or androgen-independence of the prostate cancer. Compounds that modulate the expression of the target gene or the activity of the target gene product can be used in the treatment of prostate cancer. Still further, compounds that modulate the expression of the target gene or activity of the target gene product can be used in treatments to deter benign cells from developing into prostate cancer cells. Still further, compounds that modulate the expression of the target gene or activity of the target gene product can be used to design a preventive intervention in pre-neoplastic cells in individuals at high risk.

An androgen-dependent prostate cancer cell is a cell that requires androgen for continued cell division. Conversely, and androgen-independent prostate cancer cell is a cell that can continue to divide in the absence of androgen.

Other features and advantages of the invention will be apparent from the following detailed description and claims.

### BRIEF DESCRIPTION OF THE DRAWINGS

FIG. 1 depicts the cDNA sequence (SEQ ID NO:_) and predicted amino acid sequence (SEQ ID NO:_) of human HRPCa 2.

FIG. 2 depicts the cDNA sequence (SEQ ID NO:_) and predicted amino acid sequence (SEQ ID NO:_) of human HRPCa 3.

FIG. 3 depicts the cDNA sequence (SEQ ID NO:_) and predicted amino acid sequence (SEQ ID NO:_) of human HRPCa 6/7.

FIG. 4 depicts the cDNA sequence (SEQ ID NO:_) and predicted amino acid sequence (SEQ ID NO:_) of human HRPCa 9.

FIG. 5 depicts the cDNA sequence (SEQ ID NO:_) and predicted amino acid sequence (SEQ ID NO:_) of human HRPCa 10.

FIG. 6 depicts the cDNA sequence (SEQ ID NO:_) and predicted amino acid sequence (SEQ ID NO:_) of human HRPCa 13.

FIG. 7 depicts the cDNA sequence (SEQ ID NO:_) and predicted amino acid sequence (SEQ ID NO:_) of human HRPCa 14.

FIG. 8 depicts the cDNA sequence (SEQ ID NO:_) and predicted amino acid sequence (SEQ ID NO:_) of human HRPCa 15.

FIG. 9 depicts the cDNA sequence (SEQ ID NO:_) and predicted amino acid sequence (SEQ ID NO:_) of human HRPCa 19.

### DETAILED DESCRIPTION OF THE INVENTION

The present invention is based on the discovery of ten genes, HRPCa 2, HPRCa 3, HRPCa 6/7, HRPCa 8, HRPCa 9, HRPCa 10, HRPCa 13, HRPCa 14, HRPCa 15, and HRPCa 19, whose expression is testosterone inducible in androgen-dependent prostate cancer cells (LNCaP cells) and whose expression is constitutive, i.e., not testosterone inducible, in androgen-independent prostate cancer cells (LN3 LNCaP cells). Of these, HRPCa 9 and HRPCa 10 represent novel genes which are described in greater detail in U.S. Ser. No. 09/163,759, filed Sep. 30, 1998, the contents of which is hereby incorporated by reference.

Table 1 provides a summary of certain information regarding HRPCa 2, HPRCa 3, HRPCa 6/7, HRPCa 8, HRPCa 9, HRPCa 10, HRPCa 13, HRPCa 14, HRPCa 15, and HRPCa 19 including the name of the known protein or gene and Accession Numbers which can be used to obtain the sequence of the known genes.

TABLE 1
HRPCa 2, HPRCa 3, HRPCa 6/7, HRPCa 8, HRPCa 9,
HRPCa 10, HRPCa 13, HRPCa 14, HRPCa 15, and
HRPCa 19.

| | | Accession | |
|---|---|---|---|
| Clone | Protein Name | No. | FIG. |
| HrPCa 2 | Stathmin/Oncoprotein | X53305 | 1 |
| | 18 | | |
| HRPCa 3 | Tumor antigen L6 | M90657 | 2 |
| HRPCa | TMPRSS2 | U75329 | 3 |
| 6/7 | | | |
| HRPCa 8 | Basic Transcription | Z30094 | |
| | Factor 2 | | |
| HRPCa 9 | Novel | | 4 |
| HRPCa 10 | Novel | | 5 |
| HRPCa 13 | Endozepine | M42085 | 6 |
| HRPCa 14 | KIAA0095 | D13748 | 7 |
| HRPCa 15 | eIF4AI | D13748 | 8 |
| HRPCa 19 | 15- | J05594 | 9 |
| | hydroxyprostogladin | | |
| | dehydrogenase | | |

I. Isolated Nucleic Acid Molecules

HRPCa 2, HPRCa 3, HRPCa 6/7, HRPCa 8, HRPCa 9, HRPCa 10, HRPCa 13, HRPCa 14, HRPCa 15, HRPCa 19, and PBR nucleic acid molecules are useful in the various methods of the invention. Described below are various methods for the production and use of the nucleic acid molecules of the invention.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated HRPCa 9 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention can be isolated using standard molecular biology techniques and the sequence information provided herein. Using all or a portion of the nucleic acid sequences of a gene of the invention as a hybridization probe, HRPCa-9 nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, MRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to a nucleic acid of the invention can be prepared by standard synthetic techniques, e.g., using an automated DNA synthesizer.

The nucleic acid molecules of the invention can comprise only a portion of a nucleic acid sequence of a gene of the invention, for example, a fragment which can be used as a probe or primer or a fragment encoding a biologically active portion of a protein of the invention. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence a nucleic acid of the invention.

Such probes and primers are useful for measuring the expression of a gene of the invention. For example, probes based on the human HRPCa 9 nucleotide sequence can be used to detect transcripts or genomic sequences encoding the same or identical proteins. The probe comprises a label group attached thereto, e.g., a radioisotope, a fluorescent compound, an enzyme, or an enzyme co-factor. Such probes can be used as a part of a diagnostic test kit for identifying cells or tissues which mis-express a HRPCa 9 protein, such as by measuring a level of a HRPCa 9-encoding nucleic acid in a sample of cells from a subject, e.g., detecting HRPCa-9 mRNA levels or determining whether a genomic HRPCa 9 gene has been mutated or deleted.

In addition to the nucleotide sequences disclosed herein, it will be appreciated by those skilled in the art that DNA sequence polymorphisms that lead to changes in the amino acid sequence encoded by a gene of the invention may exist within a population (e.g., the human population). Such genetic polymorphisms may exist among individuals within a population due to natural allelic variation. An allele is one of a group of genes which occur alternatively at a given genetic locus. Such natural allelic variations can typically result in 1-5% variance in the nucleotide sequence of the HRPCa 9 gene. Alternative alleles can be identified by sequencing the gene of interest in a number of different individuals. This can be readily carried out by using hybridization probes to identify the same genetic locus in a variety of individuals. The presence of a given allele may be diagnostic for prostate cancer.

Nucleic acid molecules which hybridize under stringent conditions to a gene of the invention are useful diagnostically and therapeutically. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example: of stringent hybridization conditions are hybridization in 6X sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of a gene of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

An isolated nucleic acid molecule encoding a protein having a sequence which differs from that encoded by a gene of the invention can be created by introducing one or more nucleotide substitutions, additions or deletions into the nucleotide sequences disclosed herein such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue is preferably replaced with another amino acid residue from the same side chain family. Alternatively, mutations can be introduced randomly along all or part of a coding sequence, such as by saturation mutagenesis, and the resultant mutants can be screened for biological activity to identify mutants that retain activity. Following mutagenesis, the encoded protein can be expressed recombinantly and the activity of the protein can be determined.

Antisense nucleic acid molecules, i.e., molecules which are complementary to a sense nucleic acid encoding a protein, e.g., complementary to the coding strand of a double-stranded cDNA molecule or complementary to an mRNA sequence, are useful in the methods of the invention. The antisense nucleic acid can be complementary to an entire coding strand of a gene of the invention, or to only a portion thereof, e.g., all or part of the protein coding region (or open reading frame). An antisense nucleic acid molecule can be antisense to a noncoding region of the coding strand. The noncoding regions (“5′ and 3′ untranslated regions”) are the 5′ and 3′ sequences which flank the coding region and are not translated into amino acids.

Given the coding strand sequences disclosed herein, antisense nucleic acids of the invention can be designed according to the rules of Watson and Crick base pairing. For example, the antisense oligonucleotide can be complementary to the region surrounding the translation start site of an mRNA produced by a gene of the invention. An antisense oligonucleotide can be, for example, about 5, 10, 15, 20, 25, 30, 35, 40, 45 or 50 nucleotides in length and can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.: Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In a therapeutic context, an antisense nucleic acid molecule is typically administered to a subject or generated in situ such that they hybridize with or bind to cellular MRNA and/or genomic DNA encoding a protein of the invention to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or-antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Ribozymes are also useful in the methods of the invention, e.g., to decrease expression of a protein of the invention. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave an mRNA transcript produced by a gene of the invention to thereby inhibit translation of the mRNA. A ribozyme having specificity for a given nucleic acid can be designed based upon the nucleotide sequences disclosed herein. For example, a derivative of a Tetrahymena L-19 IVS RNA can be constructed in which the nucleotide sequence of the active site is complementary to the nucleotide sequence to be cleaved in a HRPCa 9-encoding mRNA. See, e.g., Cech et al. U.S. Pat. No. 4,987,071; and Cech et al. U.S. Pat. No. 5,116,742. Alternatively, HRPCa 9 mRNA can be used to select a catalytic RNA having a specific ribonuclease activity from a pool of RNA molecules. See, e.g., Bartel and Szostak (1993) Science 261:1411-1418.

Also useful in the methods of the invention are nucleic acid molecules which form triple helical structures. For example, HRPCa 9 gene expression can be inhibited by targeting nucleotide sequences complementary to the regulatory region of the HRPCa-9 (e.g., the HRPCa 9 promoter and/or enhancers) to form triple helical structures that prevent transcription of the HRPCa 9 gene in target cells. See generally, Helene (1991) Anticancer Drug Des. 6(6):569-84; Helene (1992) Ann. N.Y. Acad. Sci. 660:27-36; and Maher (1992) Bioassays 14(12):807-15.

Nucleic acid molecules that are modified at the base moiety, sugar moiety or phosphate backbone to improve, e.g., the stability, hybridization, or solubility of the molecule are useful in the methods of the invention. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996) supra; or as probes or primers for DNA sequence and hybridization (Hyrup (1996) supra; Perry-O'Keefe et al. (1996) Proc. Natl. Acad. Sci. USA 93: 14670-675).

PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of HRPCa 9 can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) supra). The synthesis of PNA-DNA chimeras can be performed as described in Hyrup (1996) supra and Finn et al. (1996) Nucleic Acids Research 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acid Res. 17:5973-88). PNA monomers are then coupled in a stepwise manner to produce a chimeric molecule with a 5′ PNA segment and a 3′ DNA segment (Finn et al. (1996) Nucleic Acids Research 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

The oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

II. Isolated Proteins and Antibodies

Isolated proteins or the invention, and biologically active portions thereof, as well as polypeptide fragments suitable for use as immunogens to raise antibodies directed against a protein of the invention are useful in the diagnostic and therapeutic methods of the invention. Native proteins of the invention can be isolated from cells or tissue sources by an appropriate purification scheme using standard protein purification techniques. Proteins of the invention can also be produced by recombinant DNA techniques or chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations in which the protein is separated from cellular components of the cells from which it is isolated or recombinantly produced. Thus, a protein that is substantially free of cellular material includes preparations of the protein having less than about 30%, 20%, 10%, or 5% (by dry weight) of other proteins (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or other proteins or polypeptides.

A biologically active portion of a protein of the invention can be useful in screening assays. Such biologically active portions include peptides comprising amino acid sequences sufficiently identical to or derived from the native amino acid, which include fewer amino acids than the full length protein, and exhibit at least one activity of the full length protein.

Moreover, other biologically active portions, in which other regions of the protein are deleted, can be prepared by recombinant techniques and evaluated for one or more of the functional activities of the native protein.

Useful proteins are proteins which include an amino acid sequence at least about 45%, preferably 55%, 65%, 75%, 85%, 95%, or 99% identical to the amino acid sequence of a protein of the invention and retain a functional activity of a protein of the invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of overlapping positions×100). Preferably, the two sequences are the same length.

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to HRPCa-9 nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

Chimeric or fusion proteins which include all or a portion of a protein of the invention are useful in screening assays and therapeutically.

One useful fusion protein is a GST fusion protein in which all or a portion of a protein of the invention is fused to the C-terminus of the GST sequences.

Immunoglobulin fusion proteins in which all or part of a protein of the invention is fused to sequences derived from a member of the immunoglobulin protein family are useful therapeutically. An immunoglobulin fusion protein can be incorporated into pharmaceutical compositions and administered to a subject to inhibit an interaction between a protein of the invention and a protein with which it normally interacts. Such inhibition may be useful therapeutically for the treatment of prostate cancer. Moreover, immunoglobulin fusion proteins can be used as immunogens to produce antibodies in a subject, to purify ligands and in screening assays to identify molecules which inhibit the interaction of a protein of the invention and a protein or other molecule with which it normally interacts.

Chimeric or fusion proteins can be produced by standard techniques (see, e.g., Current Protocols in Molecular Biology, Ausubel et al. eds., John Wiley & Sons: 1992). Moreover, many expression vectors are commercially available that already encode a fusion moiety (e.g., a GST polypeptide). A nucleic acid encoding a polypeptide of interest can be cloned into such an expression vector such that the fusion moiety is linked in-frame to the polypeptide.

Variants of the proteins of the invention which function as either agonists (mimetics) or, preferably, as antagonists of a protein of the invention are useful therapeutically. Variants can be generated by mutagenesis, e.g., discrete point mutation or truncation. An agonist of a protein can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein.

Variants of a protein which function as either an agonist (mimetic) or antagonist can be identified by screening combinatorial libraries of mutants, e.g., truncation mutants, for protein agonist or antagonist activity. A variegated library of can be generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display) containing the set of variant protein sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential protein sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a protein coding sequence can be used to generate a variegated population of fragments for screening and subsequent selection of variants. A library of coding sequence fragments can be generated by treating a double stranded PCR fragment of protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of a protein of the invention. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants (Arkin and Yourvan (1992) Proc. Natl. Acad. Sci. USA 89:7811-7815; Delgrave et al. (1993) Protein Engineering 6(3):327-331).

An isolated protein of the invention, or a portion or fragment thereof, can be used as an immunogen to generate antibodies that bind to the protein using standard techniques for polyclonal and monoclonal antibody preparation. Such antibodies are useful therapeutically (e.g., for inhibiting protein activity) and diagnostically. A full-length protein or an antigenic fragment thereof can be used as an immunogens. Preferred antigenic peptides comprises at least 8 (preferably 10, 15, 20, or 30) amino acid residues of the protein of interest and encompass an epitope of the protein such that an antibody raised against the peptide forms a specific immune complex with the protein.

An immunogen typically is used to prepare antibodies by immunizing a suitable subject, (e.g., rabbit, goat, mouse or other mammal) with the immunogen. An appropriate immunogenic preparation can contain, for example, recombinantly expressed protein or a chemically synthesized polypeptide. The preparation can further include an adjuvant, such as Freund's complete or incomplete adjuvant, or similar immunostimulatory agent. Immunization of a suitable subject with an immunogenic preparation induces a polyclonal antibody response.

The term “antibody” as used herein refers to immunoglobulin molecules and immunologically active portions of immunoglobulin molecules, i.e., molecules that contain an antigen binding site which specifically binds an antigen, such as HRPCa 9, i.e., binds HRPCa 9, but does not substantially bind other molecules in a sample, e.g., a biological sample, which contains HRPCa 9. Examples of immunologically active portions of immunoglobulin molecules include F(ab) and F(ab′)₂ fragments which can be generated by treating the antibody with an enzyme such as pepsin.

Polyclonal antibodies can be prepared as described above by immunizing a suitable subject with a suitable immunogen. The antibody titer in the immunized subject can be monitored over time by standard techniques, such as with an enzyme linked immunosorbent assay (ELISA) using an immobilized form of the protein of interest. If desired, the antibody molecules can be isolated from the mammal (e.g., from the blood) and further purified by well-known techniques, such as protein A chromatography to obtain the IgG fraction. At an appropriate time after immunization, e.g., when the anti- HRPCa-9 antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing various antibodies, monoclonal antibody hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with the immunogen as described above, and the culture supernatants of the resulting hybridoma cells are screened to identify a hybridoma producing a monoclonal antibody that binds to the protein of interest.

Any of the many well known protocols used for fusing lymphocytes and immortalized cell lines can be applied for the purpose of generating a monoclonal antibody directed against a polypeptide of the invention (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention-are detected by screening the hybridoma culture supernatants for antibodies that bind HRPCa-9, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the protein of interest to thereby isolate immunoglobulin library members that bind the protein of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734.

Also useful in the methods of the invention are recombinant antibodies, such as chimeric and humanized monoclonal antibodies, comprising both human and non-human portions, which can be made using standard recombinant DNA techniques. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

An antibody (e.g., monoclonal antibody) directed against a polypeptide of the invention can be used to isolate a polypeptide of the invention by standard techniques, such as affinity chromatography or immunoprecipitation and to detect the polypeptide (e.g., in a cellular lysate or cell supernatant) in order to evaluate the abundance and pattern of expression of the polypeptide. Thus, antibodies directed against a polypeptide of the invention can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I , ¹³¹I, ³⁵S or ³H.

III. Recombinant Expression Vectors and Host Cells

Vectors, preferably expression vectors, containing a nucleic acid encoding protein of the invention or a portion thereof are useful in the methods of the invention. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operatively linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

Useful recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., HRPCa 9 proteins, mutant forms of HRPCa 9, fusion proteins, etc.).

The recombinant expression vectors of the invention can be designed for expression of a polypeptide of the invention in prokaryotic or eukaryotic cells, e.g., bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (l990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al.. (1987) Gene 54:113-123), pYES2 (Invitrogen Corporation, San Diego, Calif.), and picz (InVitrogen Corp, San Diego, Calif.).

Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. (supra).

Certain recombinant mammalian expression vector are capable of directing expression of a nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Recombinant expression vector comprising a nucleic acid of the invention cloned into the expression vector in an antisense orientation are useful therapeutically. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Host cells into which a recombinant expression vector capable of directing expression of a protein if the invention has been introduced are useful in the screening methods of the invention.

A host cell can be any prokaryotic or eukaryotic cell, for example, a protein of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding HRPCa-9 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a protein of the invention using standard techniques.

Certain host cells can be used to produce nonhuman transgenic animals. For example, a host cell of the invention is a fertilized oocyte or an embryonic stem cell into which HRPCa 9-coding sequences-have been introduced. Such host cells can then be used to create non-human transgenic animals in which exogenous HRPCa 9 sequences have been introduced into their genome-or homologous recombinant animals in which endogenous HRPCa 9 sequences have been altered. Such animals are useful for studying the function and/or activity of HRPCa 9 and for identifying and/or evaluating modulators of HRPCa 9 activity. The transgenic animal is preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic expressing a protein of the invention can be created by introducing nucleic acid sequence encoding the protein of interest into the-male pronuclei of a fertilized oocyte, e.g., by microinjection, retroviral infection, and allowing the oocyte to develop in a pseudopregnant female foster animal. A tissue-specific regulatory sequence(s) can be operably linked to the transgene to direct expression of the protein to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA produced by the transgene in the cells of the animals. A transgenic founder animal can then be used to breed additional animals carrying the transgene. Moreover, transgenic animals carrying a given transgene can further be bred to other transgenic animals carrying other transgenes.

To create an homologous recombinant animal, a vector is prepared which contains at least a portion of a gene of interest (e.g., the human HRPCa 9 gene or a murine HRPCa 9 gene) into which a deletion, addition or substitution has been introduced to thereby alter, e.g., functionally disrupt, the gene. The vector can be designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

Transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxp recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The nucleic acids and polypeptides of the invention as well as compounds which are modulators of the expression or activity of a protein of the invention can be incorporated into pharmaceutical compositions suitable for therapeutic or prophylactic use. Such compositions typically comprise the nucleic acid molecule, protein, or other compound and a pharmaceutically acceptable carrier. The invention includes methods for preparing pharmaceutical compositions for modulating the expression or activity of HRPCa 2, HPRCa 3, HRPCa 6/7, HRPCa 8, HRPCa 9, HRPCa 10, HRPCa 13, HRPCa 14, HRPCa 15, or HRPCa 19. Such methods comprise formulating a pharmaceutically acceptable carrier with an agent which modulates expression or activity of HRPCa 2, HPRCa 3, HRPCa 6/7, HRPCa 8, HRPCa 9, HRPCa 10, HRPCa 13, HRPCa 14, HRPCa 15, or HRPCa 19. Such compositions can further include an anti-androgenic compound. Thus, the invention further includes methods for preparing a pharmaceutical composition by formulating a pharmaceutically acceptable carrier with an agent which modulates expression or activity of HRPCa 2, HPRCa 3, HRPCa 6/7, HRPCa 8, HRPCa 9, HRPCa 10, HRPCa 13, HRPCa 14, HRPCa 15, or HRPCa 19 and an anti-androgenic agent.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a HRPCa-9 protein or anti-HRPCa-9 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of-such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can-be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. General Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenomics); and c) methods of treatment (e.g., therapeutic and prophylactic).

This invention further pertains to novel agents identified by the screening assays and uses thereof for treatments as described herein.

A. Screening Assays

The invention provides methods (also referred to herein as “screening assays”) for identifying modulators, i.e., candidate or test compounds or agents (e.g., peptides, peptidomimetics, small molecules or other drugs) which bind to a protein of the invention or have an modulatory effect on the expression or activity of a protein of the invention.

Modulators which decrease the expression or activity of a protein of the invention may be useful for the treatment of prostate cancer. Such modulators may slow the growth of androgen-independent (or androgen-dependent) prostate cancer and/or cause an androgen-independent prostate cancer to become androgen-dependent.

In one embodiment, the invention provides assays for screening candidate or test compounds which bind to or modulate the activity of the membrane-bound form of a protein or polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

In one embodiment, an assay is a cell-based assay in which a cell which expresses a membrane-bound form of a protein of the invention or a biologically active portion thereof, on the cell surface is contacted with a test compound and the ability of the test compound to bind to the protein determined. The cell, for example, can be a yeast cell or a cell of mammalian origin. Determining the ability of the test compound to bind to the protein can be accomplished, for example, by coupling the test compound with a radioisotope or enzymatic label such that binding of the test compound to the protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a protein of the invention, or a biologically active portion thereof, on the cell surface with a known compound which binds the protein to form an assay mixture, contacting the assay mixture with a test compound, and determining the ability of the test compound to interact with the protein, wherein determining the ability of the test compound to interact with the protein comprises determining the ability of the test compound to preferentially bind to the protein or a biologically active portion thereof as compared to the known compound.

In another embodiment, an assay is a cell-based assay comprising contacting a cell expressing a protein of the invention, or a biologically active portion thereof, on the cell surface with a test compound and determining the ability of the test compound to modulate (e.g., stimulate or inhibit) the activity of the protein or biologically active portion thereof. Determining the ability of the test compound to modulate the activity of the protein or a biologically active portion thereof can be accomplished, for example, by determining the ability of the protein to bind to or interact with a target molecule of the protein, i.e., a molecule with which the protein binds or interacts in nature.

Determining the ability of a protein of the invention to bind to or interact with a molecule can be accomplished by one of the methods described above for determining direct binding. In a preferred embodiment, determining the ability of the protein to bind to or interact with a target molecule can be accomplished by determining the activity of the target molecule. For example, the activity of the target molecule can be determined by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a regulatory element responsive to the protein operatively linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a protein of the invention or biologically active portion thereof with a test compound and determining the ability of the test compound to bind to the protein or biologically active portion thereof. Binding of the test compound to the protein can be determined either directly or indirectly as described above. In a preferred embodiment, the assay includes contacting the protein or biologically active portion thereof with a known compound which binds the protein to form an assay mixture, contacting the assay mixture with a test compound, and determining the ability of the test compound to interact with the protein, wherein determining the ability of the test compound to interact with the protein comprises determining the ability of the test compound to preferentially bind to the protein or biologically active portion thereof as compared to the known compound.

In another embodiment, an assay is a cell-free assay comprising contacting a protein of the invention or biologically active portion thereof with a test compound and determining the ability of the test compound to modulate (e.g., stimulate or inhibit) the activity of the protein or biologically active portion thereof. Determining the ability of the test compound to modulate the activity of the protein can be accomplished, for example, by determining the ability of the protein to bind to a target molecule by one of the methods described above for determining direct binding. In an alternative embodiment, determining the ability of the test compound to modulate the activity of the protein can be accomplished by determining the ability of the protein to further modulate a target molecule. For example, the catalytic/enzymatic activity of the target molecule on an appropriate substrate can be determined as previously described.

In yet another embodiment the cell-free assay comprises contacting a protein of the invention or a biologically active portion thereof with a known compound which binds to the protein to form an assay mixture, contacting the assay mixture with a test compound, and determining the ability of the test compound to interact with the potein, wherein determining the ability of the test compound to interact with the protein comprises determining the ability of the protein to preferentially bind to or modulate the activity of a target molecule of the protein.

The cell-free assays of the present invention are amenable to use of both the soluble form or the membrane-bound form of a protein of the invention. In the case of cell-free assays comprising the membrane-bound form of a protein of the invention, it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Tsotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either a protein of the invention or its target molecule to facilitate separation of complexed from uncomplexed forms of one or both of the proteins, as well as to accommodate automation of the assay. Binding of a test compound to a protein of the invention, or the protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/protein of the invention fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or a protein of the invention, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the protein of the invention can be determined using standard techniques.

Other techniques for immobilizing proteins on matrices can also be used in the screening assays of the invention. For example, either a protein of the invention or its target molecule can be immobilized utilizing conjugation of biotin and streptavidin. Biotinylated molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with a protein of the invention or a target molecule but which do not interfere with binding of the protein of the invention to its target molecule can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein of the invention or target molecule, as well as enzyme-linked assays which rely on detecting an enzymatic activity associated with a protein of the invention or its target molecule.

In another embodiment, modulators of expression of a protein of the invention are identified in a method in which a cell is contacted with a candidate compound and the expression of the mRNA or protein of the invention in the cell is determined. The level of expression of mRNA or protein of the invention in the presence of the candidate compound is compared to the level of expression in the absence of the candidate compound. The candidate compound can then be identified as a modulator of expression based on this comparison. For example, when expression of the mRNA or protein of the invention is greater. (statistically significantly greater) in the presence of the candidate compound than in its absence, the candidate compound is identified as a stimulator of mRNA or protein expression. Alternatively, when expression of the MRNA or protein of the invention is less (statistically significantly less) in the presence of the candidate compound than, in its absence, the candidate compound is identified as an inhibitor of mRNA or protein expression. The level of expression of an mRNA or protein of the invention can be determined by methods described herein.

In yet another aspect of the invention, a protein of the invention can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the protein and modulate activity of the protein.

In another embodiment, modulators of activity of a protein of the invention are identified in a method in which a cell is contacted with a candidate compound and the activity of a protein of the invention in the cell is determined. The level of activity of the protein of the invention in the presence of the candidate compound is compared to the level of expression in the absence of the candidate compound. The candidate compound can then be identified as a modulator of activity based on this comparison. For example, when the activity of a protein of the invention is greater (statistically significantly greater) in the presence of the candidate compound than in its absence, the candidate compound is identified as a stimulator protein activity. Alternatively, when the activity of a protein of the invention is less (statistically significantly less) in the presence of the candidate compound than in its absence, the candidate compound is identified as an inhibitor of protein activity. Such inhibitors are useful for the treatment of androgen-dependent and androgen-independent prostate cancers. The level of activity of a protein of the invention can be determined by an method used to measure activity of the protein.

This invention further pertains to novel agents identified by the above-described-screening assays and uses thereof for treatments as described herein.

B. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic and prognostic (or predictive) assays for determining the expression or activity of a protein of invention, in the context of a biological sample (e.g., prostate cells or tissue) to thereby determine whether an individual is afflicted with prostate cancer, or is at risk of developing prostate cancer. (or another cancer). The methods are particularly useful for determining whether an individual is at risk for developing an androgen-independent prostate cancer. Such assays can be used for prognostic or predictive purpose to thereby prophylactically treat an individual prior to the onset of prostate cancer.

Another aspect of the invention provides methods for determining the expression of a nucleic acid or protein of the invention or the activity of a protein of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression of a nucleic acid or protein of the invention or the activity of a protein of the invention in clinical trials.

These and other agents are described in further detail in the following sections.

1. Diagnostic Assays

An exemplary method for detecting the presence or absence of a protein or nucleic acid of the invention in biological sample involves obtaining a biological sample from a test subject and contacting the biological sample with a compound or an agent capable of detecting the protein or nucleic acid (e.g., mRNA,.genomic DNA) such that the presence of the protein or nucleic acid is detected in the biological sample. A preferred agent for detecting MRNA or genomic DNA is a labeled nucleic acid probe capable of hybridizing to the selected MRNA or genomic DNA. The nucleic acid probe can be, for example, a full-length nucleic acid of the invention or a portion thereof, such as an oligonucleotide of at least 15, 30, 50, 100, 250 or 500 nucleotides in length and sufficient to specifically hybridize under stringent conditions to the selected mRNA or genomic DNA; Other suitable probes for use in the diagnostic assays of the invention are described herein.

A preferred agent for detecting a protein of the invention is an antibody capable of binding to the protein, preferably an antibody with a detectable label. Antibodies can be polyclonal, or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells (e.g., prostate cells) and biological fluids isolated from a subject, as well as tissues, cells and fluids present within a subject. That is, the detection method of the invention can be used to detect a nucleic acid or protein of the invention in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody directed against a protein of the invention. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a protein or nucleic acid of the invention, such that the presence of the protein or nucleic acid is detected in the biological sample, and comparing the presence of the protein or nucleic acid of the invention in the control sample with the presence of the protein or nucleic acid of the invention in the test sample.

The invention also encompasses kits for detecting the presence of a protein or nucleic acid of the invention in a biological sample (a test sample). Such kits can be used to determine if a subject is suffering from or is at increased risk of developing prostate cancer (e.g., an androgen-independent prostate cancer). For example, the kit can comprise a labeled compound or agent capable of detecting a protein or nucleic acid of the invention in a biological sample and means for determining the amount of the protein or nucleic acid of the invention in the sample (e.g., an antibody or an oligonucleotide probe). Kits may also include instruction for observing that the tested subject is suffering from or is at risk of developing a prostate cancer if the amount of the protein or nucleic acid of the invention is above a normal level or is constitutively expressed in the absence of testosterone or another androgen.

For antibody-based kits, the kit may comprise, for example: (1) a first antibody (e.g., attached to a solid support) which binds to the protein of the invention; and, optionally, (2) a second, different antibody which binds to the protein or the first antibody and is conjugated to a detectable agent.

For oligonucleotide-based kits, the kit may comprise, for example: (1) an oligonucleotide, e.g., a detectably labeled oligonucleotide, which hybridizes to a nucleic acid of the invention or (2) a pair of primers useful for amplifying a nucleic acid of the invention.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing prostate cancer (e.g., androgen-independent prostate cancer).

2. Prognostic Assays

The methods described herein can furthermore be utilized as diagnostic or prognostic assays to identify subjects having or at risk of developing a disease or disorder associated with aberrant expression or activity of HRPCa 2, HPRCa 3, HRPCa 6/7, HRPCa 8, HRPCa 9, HRPCa 10, HRPCa 13, HRPCa 14, HRPCa 15, or HRPCa 19. For example, the assays described herein, such as the preceding diagnostic assays or the following assays, can be utilized to identify a subject having or at risk of developing a disorder associated with HRPCa 2, HPRCa 3, HRPCa 6/7, HRPCa 8, HRPCa 9, HRPCa 10, HRPCa 13, HRPCa 14, HRPCa 15, or HRPCa 19 protein, nucleic acid expression (e.g., constitutive expression) or activity, e.g., androgen-independent prostate cancer. Alternatively, the prognostic assays can be utilized to identify a subject having or at risk for developing such a disease or disorder. Thus, the present invention provides a method in which a test sample (e.g., prostate cells) is obtained from a subject and a protein or nucleic acid (e.g., mRNA, genomic DNA) of the invention is detected, wherein the presence of increased or constitutive expression of protein or nucleic acid of the invention is diagnostic for a subject having or at risk of developing prostate cancer (e.g., androgen-independent prostate cancer). As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) prostate cancer. For example, such methods can be used to determine whether a subject can be effectively treated with a specific agent or class of agents (e.g., agents of a type which decrease activity of a protein of the invention or agents of a type which decrease expression of a protein or nucleic acid of the invention). Thus, the present invention provides methods for determining whether a subject can be effectively treated with an agent for a disorder (e.g., prostate cancer) associated with aberrant expression (e.g., constitutive or non-androgen inducible expression) or activity of HRPCa 2, HPRCa 3, HRPCa 6/7, HRPCa 8, HRPCa 9, HRPCa 10, HRPCa 13, HRPCa 14, HRPCa 15, or HRPCa 19 in which a test sample is obtained and HRPCa 2, HPRCa 3, HRPCa 6/7, HRPCa. 8, HRPCa 9, HRPCa 10, HRPCa 13, HRPCa 14, HRPCa 15, or HRPCa 19 protein or nucleic acid is detected.

The methods of the invention can also be used to detect genetic lesions or mutations in a gene of the invention, thereby determining if a subject with the lesioned gene is at risk for a disorder associated with such a lesion. In preferred embodiments, the methods include detecting, in a sample of cells from the subject, the presence or absence of a genetic lesion or mutation characterized by at least one of an alteration affecting the integrity of a gene of the invention, or the mis-expression of a protein or nucleic acid of the invention. For example, such genetic lesions can be detected by ascertaining the existence of at least one of: 1) a deletion of one or more nucleotides from a gene of the invention; 2) an addition of one or more nucleotides to a gene of the invention; 3) a substitution of one or more nucleotides of a gene of the invention; 4) a chromosomal rearrangement of a gene of the invention; 5) an alteration in the level of a messenger RNA transcript of a gene of the invention, 6) aberrant modification of a gene of the invention, such as of the methylation pattern of the genomic DNA, 7) the presence of a non-wild type splicing pattern of a messenger RNA transcript of a gene of the invention, 8) a non-wild type level of a protein of the invention, 9) allelic loss of a gene of the invention, 10) inappropriate post-translational modification of a protein of the invention, 11) loss of androgen-inducibility of a gene of the invention. As described herein, there are a large number of assay techniques known in the art which can be used for detecting lesions in a gene of the invention. A preferred biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a gene of the invention under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a gene of the invention from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a gene of the invention can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in a gene of the invention can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the Gene of the invention and detect mutations by comparing the sequence of the sample with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the gene of the invention include methods in which protection from cleavage agents is used to detect mismatched bases in RNA/RNA or RNA/DNA heteroduplexes (Myers et al. (1985) Science 230:1242). In general, the technique of “mismatch cleavage” entails providing heteroduplexes formed by hybridizing (labeled) RNA or DNA containing the wild-type the sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al (1988) Proc. Natl. Acad. Sci. USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in a cDNA obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on the sequence of a gene of the invention, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in a gene of the invention For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet Anal Tech Appl 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene of the invention, e.g., prostate cancer.

Furthermore, any cell type or tissue, preferably prostate cells, in which HRPCa 2, HPRCa 3, HRPCa 6/7, HRPCa 8, HRPCa 9, HRPCa 10, HRPCa 13, HRPCa 14, HRPCa 15, or HRPCa 19 is expressed may be utilized in the prognostic assays described herein.

3. Pharmacogenomics

Agents, or modulators which have an inhibitory effect on the expression of a nucleic acid or protein of the invention or the activity of a protein of the invention as identified by a screening assay described herein can be administered to individuals to treat (prophylactically or therapeutically) prostate cancer. In conjunction with such treatment, the pharmacogenbmics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a protein of the invention or the expression of a nucleic acid or protein of the invention or the mutation content of a gen of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” These pharmacogenetic conditions can occur either as rare defects or as polymorphisms.

Thus, the activity of a protein of the invention or the expression of a nucleic acid or protein of the invention or the mutation content of a gene of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of the expression or activity of HRPCa 2, HPRCa 3, HRPCa 6/7, HRPCa 8, HRPCa 9, HRPCa 10, HRPCa 13, HRPCa 14, HRPCa 15, or HRPCa 19 such as a modulator identified by one of the exemplary screening assays described herein.

4. Monitoring of Effects During Clinical Trials

Monitoring the influence of agents (e.g., drugs, compounds) on the expression or activity of HRPCa 2, HPRCa 3, HRPCa 6/7, HRPCa 8, HRPCa 9, HRPCa 10, HRPCa 13, HRPCa 14, HRPCa 15, or HRPCa 19 can be applied not only in basic drug screening, but also in clinical trials. For example, the effectiveness of an agent, as determined by a screening assay, to decrease expression or activity of HRPCa 2, HPRCa 3, HRPCa 6/7, HRPCa 8, HRPCa 9, HRPCa 10, HRPCa 13, HRPCa 14, HRPCa 15, or HRPCa 19, can be monitored in clinical trials of subjects exhibiting increased expression (or constitutive expression) or increase activity of HRPCa 2, HPRCa 3, HRPCa 6/7, HRPCa 8, HRPCa 9, HRPCa 10, HRPCa 13, HRPCa 14, HRPCa 15, or HRPCa 19.

For example, in a clinical trial, cells can be isolated and RNA prepared and analyzed for the levels of expression of HRPCa 2, HPRCa 3, HRPCa 6/7, HRPCa 8, HRPCa 9, HRPCa 10, HRPCa 13, HRPCa 14, HRPCa 15, or HRPCa 19 (or various combinations thereof). The levels of gene expression (i.e., a gene expression pattern) can be quantified by Northern blot analysis or RT-PCR, as described herein, or alternatively by measuring the amount of protein produced, by one of the methods as described herein, or by measuring the levels of activity. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression or activity of HRPCa 2, HPRCa 3, HRPCa 6/7, HRPCa 8, HRPCa 9, HRPCa 10, HRPCa 13, HRPCa 14, HRPCa 15, or HRPCa 19 the preadministration sample; (iii) obtaining one or more post-administration samples from the subject; (iv) detecting the level of expression or activity of HRPCa 2, HPRCa 3, HRPCa 6/7, HRPCa 8, HRPCa 9, HRPCa 10, HRPCa 13, HRPCa 14, HRPCa 15, or HRPCa 19 in the post-administration samples; (v) comparing the level of expression or activity of HRPCa 2, HPRCa 3, HRPCa 6/7, HRPCa 8, HRPCa 9, HRPCa 10, HRPCa 13, HRPCa 14, HRPCa 15, or HRPCa 19 in the pre-administration sample with the-level of expression or activity of HRPCa 2, HPRCa 3, HRPCa 6/7, HRPCa 8, HRPCa 9, HRPCa 10, HRPCa 13, HRPCa 14, HRPCa 15, or HRPCa 19 in the post-administration sample or samples; and (vi) altering the administration of the agent to the subject accordingly. For example, increased administration of the agent may be desirable to decrease the expression or activity of HRPCa 2, HPRCa 3, HRPCa 6/7, HRPCa 8, HRPCa 9, HRPCa 10, HRPCa 13, HRPCa 14, HRPCa 15, or HRPCa 19 lower levels than detected, i.e., to increase the effectiveness of the agent.

C. Methods of Treatment

The present invention provides for both prophylactic and therapeutic methods of treating a subject at risk of (or susceptible to) a disorder or having a disorder associated with aberrant HRPCa 2, HPRCa 3, HRPCa 6/7, HRPCa 8, HRPCa 9, HRPCa 10, HRPCa 13, HRPCa 14, HRPCa 15, or HRPCa 19 expression or activity. Such disorders include prostate cancer, especially androgen-resistant prostate cancer.

1. Prophylactic Methods

In one aspect, the invention provides a method for preventing in a subject, a disease or condition associated with an aberrant HRPCa 2, HPRCa 3, HRPCa 6/7, HRPCa 8, HRPCa 9, HRPCa 10, HRPCa 13, HRPCa 14, HRPCa 15, or HRPCa 19 expression or activity, by administering to the subject an agent which modulates HRPCa 2, HPRCa 3, HRPCa 6/7, HRPCa 8, HRPCa 9, HRPCa 10, HRPCa 13, HRPCa 14, HRPCa 15, or HRPCa 19 expression or at least one HRPCa 2, HPRCa 3, HRPCa 6/7, HRPCa 8; HRPCa 9, HRPCa 10, HRPCa 13, HRPCa 14, HRPCa 15, or HRPCa 19 activity. Subjects at risk for a disease (e.g., prostate cancer) which is caused by or contributed to by aberrant HRPCa 2, HPRCa 3, HRPCa 6/7, HRPCa 8, HRPCa 9, HRPCa 10, HRPCa 13, HRPCa 14, HRPCa 15, or HRPCa 19 expression or activity can be identified by, for example, any or a combination of diagnostic or prognostic assays as described herein. Administration of a prophylactic agent can occur prior to the manifestation of symptoms characteristic of the disease or disorder such that the disease or disorder is prevented or, alternatively, delayed in its progression.

2. Therapeutic Methods

Another aspect of the invention pertains to methods of modulating HRPCa 2, HPRCa 3, HRPCa 6/7, HRPCa 8, HRPCa 9, HRPCa 10, HRPCa 13, HRPCa 14, HRPCa 15, or HRPCa 19 expression or activity for therapeutic purposes. The modulatory method of the invention involves contacting a cell with an agent that modulates one or more of the activities of HRPCa 2, HPRCa 3, HRPCa 6/7, HRPCa 8, HRPCa 9, HRPCa 10, HRPCa 13, HRPCa 14, HRPCa 15, or HRPCa 19. An agent that modulates HRPCa 2, HPRCa 3, HRPCa 6/7, HRPCa 8, HRPCa 9, HRPCa 10, HRPCa 13, HRPCa 14, HRPCa 15, or HRPCa 19 activity can be an agent as described herein, such as a nucleic acid or a protein, a naturally-occurring cognate ligand, a peptide, a peptidomimetic, or other small molecule. Useful agents inhibit one or more of the biological activities HRPCa 2, HPRCa 3, HRPCa 6/7, HRPCa 8, HRPCa 9, HRPCa 10, HRPCa 13, HRPCa 14, HRPCa 15, or HRPCa 19. Examples of such inhibitory agents include antisense nucleic acid molecules and antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of HRPCa 2, HPRCa 3, HRPCa 6/7, HRPCa 8, HRPCa 9, HRPCa 10, HRPCa 13, HRPCa 14, HRPCa 15, or HRPCa 19. In one embodiment, the method involves administering an agent (e.g., an agent identified by a screening assay described herein), or combination of agents that modulates (e.g., downregulates) HRPCa 2, HPRCa 3, HRPCa 6/7, HRPCa 8, HRPCa 9, HRPCa 10, HRPCa 13, HRPCa 14, HRPCa 15, or HRPCa 19 expression or activity.

Agents which modulate (reduce) the expression or activity of HRPCa 2, HPRCa 3, HRPCa 6/7, HRPCa 8, HRPCa 9, HRPCa 10, HRPCa 13, HRPCa 14, HRPCa 15, or HRPCa 19 can be co-administered with an anti-androgenic compound. In addition, an agent which modulates (reduces) the expression or activity of HRPCa 2, HPRCa 3, HRPCa 6/7, HRPCa 8, HRPCa 9, HRPCa 10, HRPCa 13, HRPCa 14, HRPCa 15, or HRPCa 19, optionally co-administered with an anti-androgenic compound, can be administered to a patient in conjunction with other anti-cancer therapies, e.g., other chemotherapies, radiation therapy, or anti-neovascularization therapy.

VI. Prostate Cancer

The expression level of HRPCa 2, HPRCa 3, HRPCa 6/7, HRPCa 8, HRPCa 9, HRPCa 10, HRPCa 13, HRPCa 14, HRPCa 15, or HRPCa 19 may be used to assess patients undergoing clinical evaluation for the treatment of prostate cancer. Thus, the genes of the invention can be used to select an appropriate therapy for an individual and to monitor an ongoing therapy. They may also be utilized as surrogate markers to monitor clinical human trials of a drug being tested for efficacy as a prostate cancer treatment. In either case, one or more of the genes of the invention may be utilized as a marker.

One can determine whether a given patient will benefit from anti-androgenic therapy by examining the expression level of one or more of the genes of the invention. If one or more of these genes is more highly expressed in the patient's prostate cancer cells in the presence of testosterone than in the absence of testosterone, the prostate cancer cells are more likely to respond to anti-androgenic therapy. As used herein, “anti-androgenic therapy” refers broadly to any therapy which interferes with or blocks the synthesis or action of an androgen. Similarly, “anti-testosterone therapy” refers broadly to any therapy which interferes with or blocks the synthesis or action of testosterone. Such therapies can, for example, block ligand receptor binding (e.g., binding of testosterone to its receptor), receptor signaling (e.g., signal transduction mediated by the binding of testosterone to its receptor), or the activity or expression of a downstream regulated gene.

The biological function of HRPCa 2, HPRCa 3, HRPCa 6/7, HRPCa 8, HRPCa 9, HRPCa 10, HRPCa 13, HRPCa 14, HRPCa 15, and HRPCa 19 can be more directly assessed by utilizing relevant in vivo and in vitro systems. In vivo systems can include, but are not limited to, animal systems which naturally exhibit symptoms of prostate cancer or ones which have been engineered to exhibit such symptoms.

The role of HRPCa 2, HPRCa 3, HRPCa 6/7, HRPCa 8, HRPCa 9, HRPCa 10, HRPCa 13, HRPCa 14, HRPCa 15, and HRPCa 19 can be determined by transfecting cDNAs encoding these gene products into appropriate cell lines, such as, for example, a prostate cancer cell line and analyzing the effect of the gene product on cell growth.

In further characterizing the biological function of the genes of the invention, the expression of these genes can be modulated within the in vivo and/or in vitro systems, i.e., either over-expressed or under-expressed, and the subsequent effect on the system then assayed. Alternatively, the activity of the product of the identified gene can be modulated by either increasing or decreasing the level of activity in the in vivo and/or in vitro system of interest, and assessing the effect of such modulation.

The information obtained through such characterizations can suggest relevant methods for the treatment of prostate cancer. For example, treatment can include a modulation of gene expression and/or gene product activity. Characterization procedures such as those described herein can indicate where such modulation should involve an increase or a decrease in the expression or activity of the gene or gene product of interest.

Described below are in vitro and in vivo systems which can be used to further characterize the genes of the invention. These system can also be used as part of screening strategies designed to identify compounds which are capable of preventing and/or ameliorating symptoms of prostate cancer. Thus, these systems can be used to identify drugs, pharmaceuticals, therapies and interventions which can be effective in treating prostate cancer and to determine the in vivo efficacy of drugs, pharmaceuticals, therapies and interventions.

1. In Vitro Systems

Cells that contain and express a gene of the invention and exhibit cellular phenotypes associated with prostate cancer (e.g, androgen-independent prostate cancer), can be utilized to identify compounds that exhibit an ability to prevent and/or treat prostate cancer.

Further, pattern of expression of some or all of the genes of the invention in cells of interest can be analyzed and compared to the normal expression pattern. Those compounds which cause cells exhibiting cellular phenotypes of prostate cancer to produce an expression pattern more closely resembling a normal expression pattern for the cell of interest can be considered candidates for further testing regarding an ability to ameliorate the symptoms of prostate cancer.

Cells which will be utilized for such assays can, for example, include LNCaP cells. In addition, purified primary or secondary tumor cells derived from either transgenic or non-transgenic tumor cells can be used.

Further, cells which can be used for such assays can also include recombinant, transgenic cell lines. While primary cultures derived from the metastasis in transgenic animals can be utilized, the generation of continuous cell lines is preferred. For examples of techniques which can be used to derive a continuous cell line from a transgenic animal (Small et al. (1985) Mol. Cell Biol. 5:642-648).

Alternatively, prostate cancer cells can be transfected with sequences capable of increasing or decreasing the expression of a gene of the invention within the cell. For example, a gene of the invention can be introduced into, and over expressed in, the genome of the cell of interest, or, if endogenous target gene sequences are present, they can either be overexpressed or, alternatively, be disrupted in order to underexpress or inactivate target gene expression.

In order to overexpress a target gene sequence, the coding portion of the target gene sequence can be ligated to a regulatory sequence which is capable of driving gene expression in the cell type of interest. Such regulatory regions will be well known to those of skill in the art.

For under expression of an endogenous target gene sequence, such a sequence can be isolated and engineered such that when reintroduced into the genome of the cell type of interest, the endogenous target gene alleles will be inactivated. Preferably, the engineered target gene sequence is introduced via gene targeting such that the endogenous target sequence is disrupted upon integration of the engineered target gene sequence into the cell's genome.

Transfection of target gene sequence nucleic acid can be accomplished by utilizing standard techniques (see, e.g., Ausubel, supra). Transfected cells should be evaluated for the presence of the recombinant target gene sequences, for expression and accumulation of target gene mRNA, and for the presence of recombinant target gene protein production. In instances wherein a decrease in target gene expression is desired, standard techniques can be used to demonstrate whether a decrease in endogenous target gene expression and/or in target gene product production is achieved.

2. In Vivo Systems

In vivo systems of prostate cancer can be either non-recombinant animals or recombinantly engineered transgenic animals. Such models may be generated, for example, by introducing tumor cells into syngeneic mice using techniques such as implanting prostatic cancer cells into the prostate gland. After an appropriate period of time, the tumors which result from these injections can be counted and analyzed.

For the generation of animal models of prostate cancer, cells derived from, for example, a prostate cancer cell line may be implanted into the prostate of an animal and the resulting tumors may be analyzed and compared to, for example, normal tissue.

The role of HRPCa 2, HPRCa 3, HRPCa 6/7, HRPCa 8, HRPCa 9, HRPCa 10, HRPCa 13, HRPCa 14, HRPCa 15, or HRPCa 19 can be determined by transfecting cDNAs encoding such the gene product into the appropriate cell line and analyzing its effect on the cells' ability to induce prostate cancer in animal models such as these. The role of the identified gene products may be further analyzed by, for example, culturing cells derived from the tumors which develop in the animal models, introducing these cultured cells into animals, and subsequently measuring the level of identified gene product present in the resulting tumor cells. In this manner, cell line variants are developed which can be useful in analyzing the role of quantitative and/or qualitative differences in the expression of the identified genes on the cells' ability to induce prostate cancer.

Recombinant animal models for prostate cancer can be engineered by utilizing, for example, target gene sequences such as those described herein in conjunction with techniques for producing transgenic animals that are well known to those of skill in the art. For example, target gene sequences can be introduced into, and overexpressed in, the genome of the animal of interest, or, if endogenous target gene sequences are present, they can either be overexpressed or, alternatively, can be disrupted in order to underexpress or inactivate target gene expression.

In order to overexpress a gene of the invention, the coding portion of the gene sequence can be ligated to a regulatory sequence which is capable of driving gene expression in the animal and cell type of interest. Such regulatory regions will be well known to those of skill in the art.

In order to obtain underexpression of an endogenous target gene sequence, such a sequence can be introduced into the genome of the animal of interest such that the endogenous target gene alleles will be inactivated. Preferably, an engineered sequence comprising at least part of the target gene sequence is utilized and is introduced, via gene targeting, such that the endogenous target sequence is disrupted upon integration of the engineered target gene sequence into the animal's genome.

Animals of any species, including, but not limited to, mice, rats, rabbits, guinea pigs, pigs, micro-pigs, goats, and non-human primates, e.g., baboons, monkeys, and chimpanzees can be used to generate animal models of prostate cancer.

Any technique known in the art can be used to introduce a gene of the invention into animals to produce the founder lines of transgenic animals, as described above.

Once transgenic animals have been generated, the expression of the recombinant target gene and protein can be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques which include, but are not limited to, Northern blot analysis of tissue samples obtained from the animal, in situ hybridization analysis, and RT-coupled PCR. Samples of target gene-expressing tissue, can also be evaluated immunocytochemically using antibodies specific for the transgenic product of interest.

The transgenic animals that express a recombinant nucleic acid or protein of the invention are further evaluated to identify those animals which display prostate cancer characteristics.

Additionally, specific cell types within the transgenic animals can be analyzed for cellular phenotypes characteristic of prostate cancer. Such cellular phenotypes can include, for example, differential gene expression characteristic of prostate cancer cells. Further, such cellular phenotypes can include as assessment of a particular cell type gene expression profile and its comparison to known expression profiles of the particular cell type in animals exhibiting prostate cancer. Such transgenic animals serve as suitable model systems for prostate cancer.

Once target gene transgenic founder animals are produced (i.e., those animals which express a protein of the invention in cells or tissues of interest, and which, preferably, exhibit prostate cancer characteristics), they can be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound transgenics that express the transgene of interest at higher levels because of the effects of additive expression of each transgene; crossing of heterozygous transgenic animals to produce animals homozygous for a given integration site in order to both augment expression and eliminate the possible need for screening of animals by DNA analysis; crossing of separate homozygous lines to produce compound heterozygous or homozygous lines; breeding animals to different inbred genetic backgrounds so as to examine effects of modifying alleles on expression of the target gene transgene and the development of symptoms of prostate cancer. One such approach is to cross the transgenic founder animals with a wild-type strain to produce an F1 generation that exhibits symptoms of prostate cancer. The F1 generation can then be inbred in order to develop a homozygous line, if it is found that homozygous transgenic animals are viable.

This invention is further illustrated by the following examples which should not be construed as limiting. The contents of all references, patents and published patent applications cited throughout this application are hereby incorporated by reference.

### EXAMPLES

### Example 1

### Identification of Genes that are Constitutively Expressed in Androgen-Independent Prostate Cancer Cells

HRPCa 2, HPRCa 3, HRPCa 6/7, HRPCa 8, HRPCa 9, HRPCa 10, HRPCa 13, HRPCa 14, HRPCa 15, and were identified using a strategy designed to identify genes whose expression is induced by testosterone (or a similar androgen) in androgen-dependent prostate cancer cells and are constitutively expressed in androgen-independent prostate cancer cells.

WT LNCaP cells (androgen-dependent prostate cancer cells) were routinely grown in T162 flasks coated with Matrigel in RPMI-1640 medium supplemented with 10% FBS and 50 nM testosterone.

To identify androgen regulated genes, LNCaP cells were first incubated in the absence of androgens and then treated with either testosterone or casodex. Ten T162 flasks of LNCaP cells were pre-incubated for 24.5 hours in dye-free RPMI-1640 containing 2% charcoal stripped serum. Following pretreatment, five T162 flasks of pretreated cells were treated with testosterone-containing medium (dye-free RPMI-1640, 2% CSS, 100 nM testosterone, 0.09% DMSO), and five T162 flasks of pretreated cells were treated with casodex-containing medium (dye-free RPMI-1640, 2% CSS, 100 μM casodex, 0.09% DMSO). After 25 hours of incubation in testosterone-containing medium or casodex-containing medium, the cells were detached from the flasks with trypsin and pelleted

Total RNA was prepared from the cell pellets using the RNeasy protocol (Qiagen). Approximately 260 μg of total RNA was obtained from each cell pellet. Next, polyA+ RNA was prepared form approximately 240 μg of each total RNA sample using the Oligotex protocol (Qiagen), approximately 6 μg of polyA+ RNA was obtained from each 240 μg total RNA sample, and 2 μg of each polyA+ RNA sample was used for the generation of subtraction libraries using the PCR-select protocol (Clontech; Palo Alto, Calif.).

The PCR products, representing partial cDNAs of putatively differentially expressed Minas, were subcloned into pCR2.1 (InVitrogen) and transformed into INValphaF¹ cells.

The cDNA inserts from individual clones of the subtraction libraries were PCR amplified and spotted onto nylon to generated high-density arrays. The arrays were probed with first strand cDNA from: WT LNCaP cells treated with 100 nM testosterone, WT LNCaP cells treated with 100 μM casodex, LN3 LNCaP cells (an androgen independent variant of LNCaP cells) treated with 100 nM testosterone, LN3 LNCaP cells treated with 100 μM casodex, WT LNCaP cells treated with 1 nM R1881, and WT LNCaP cells treated with stripped serum. Quantitative analysis of radioactive signal at each cDNA spot was performed to identify the relative differential expression of each cDNA among the above cell lines and drug treatments. The clones chosen for further analysis demonstrated higher expression levels in WT LNCaP cells treated with testosterone than in WT LNCaP cells treated with casodex and equal expression levels in LN3 cells treated with testosterone or casodex.

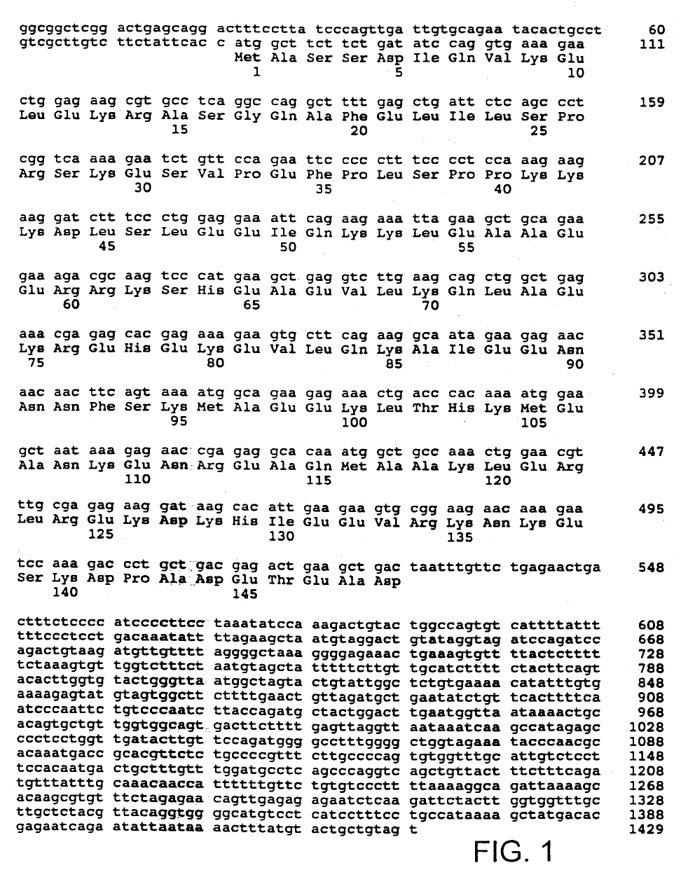

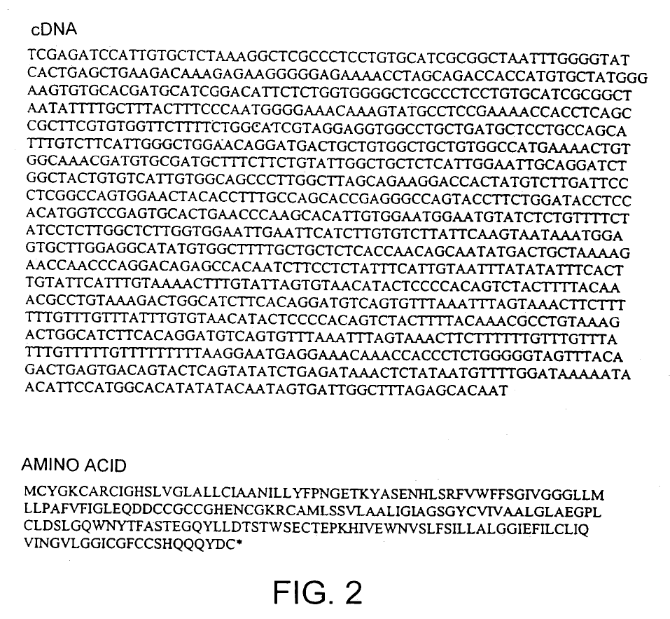

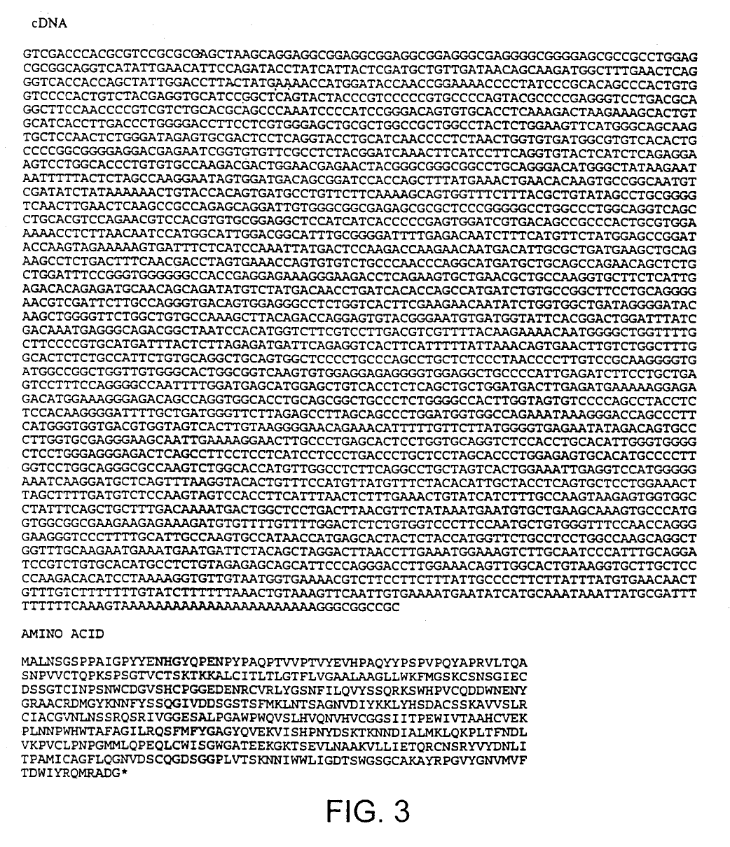

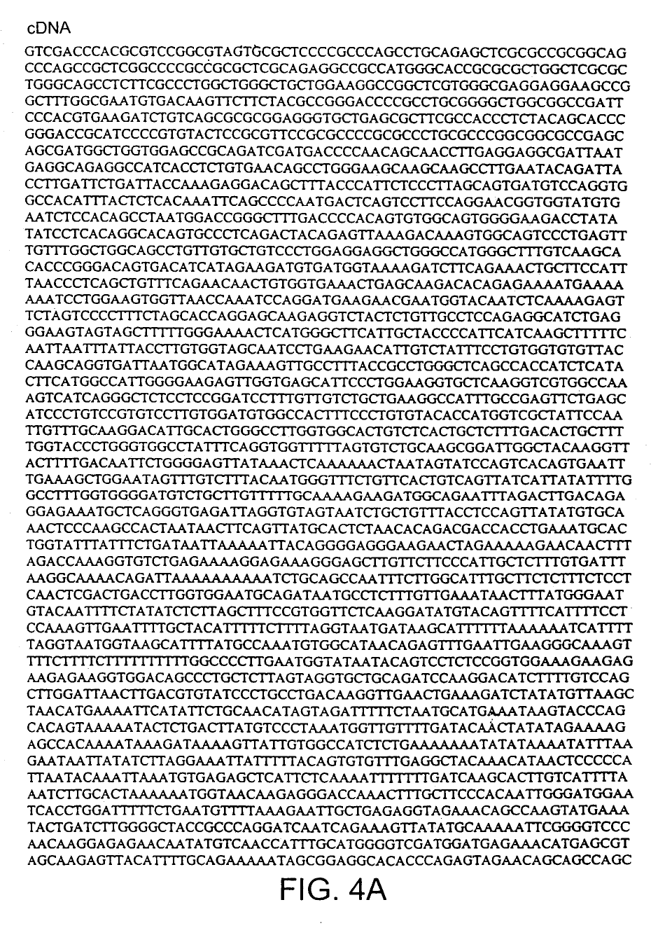

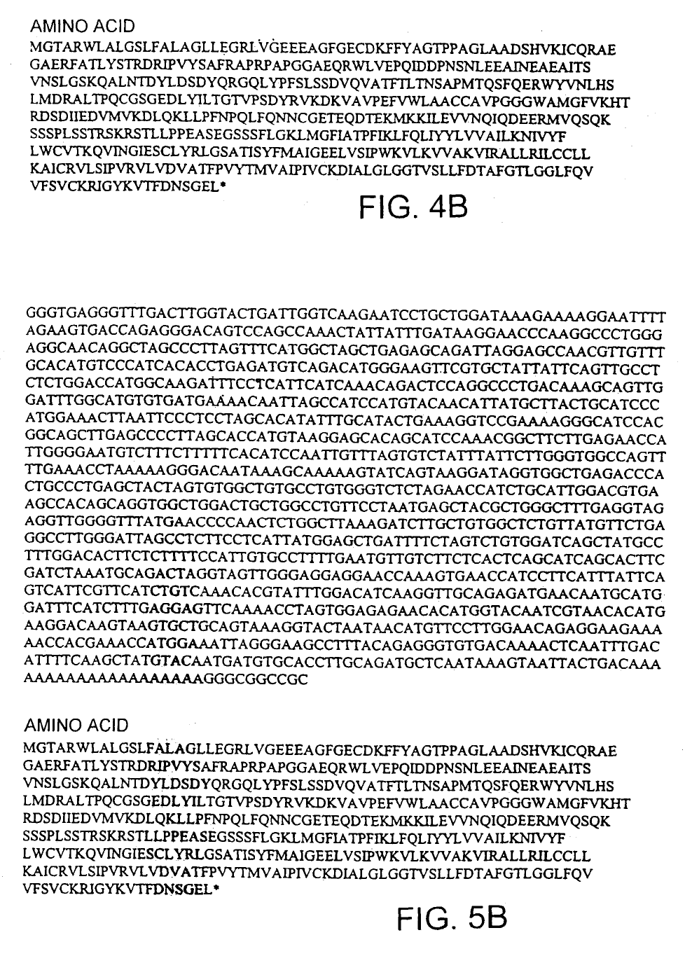

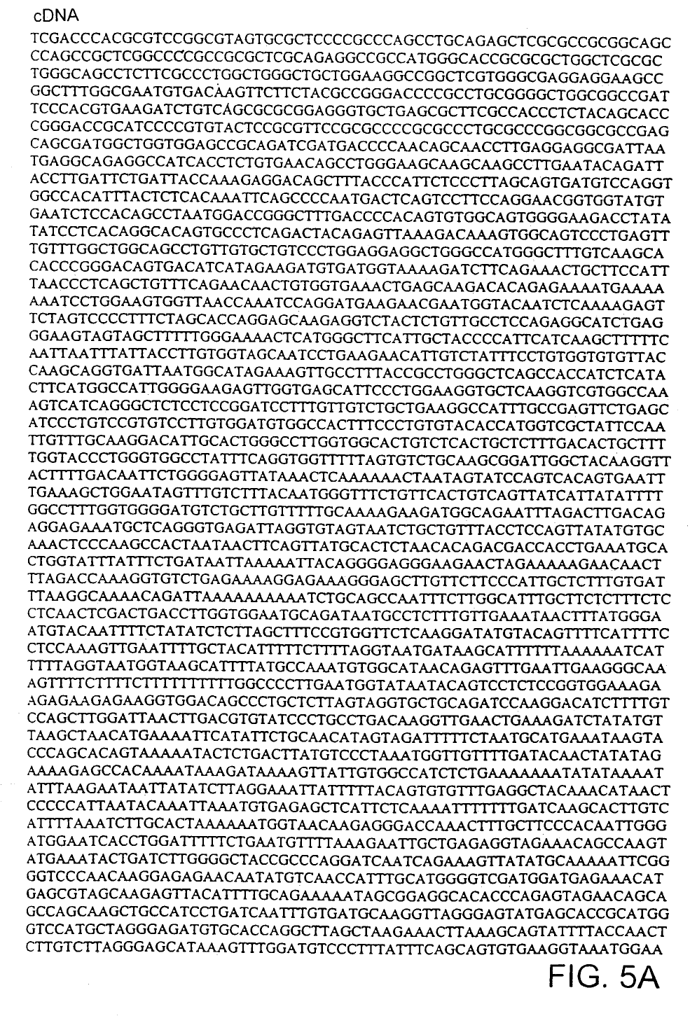

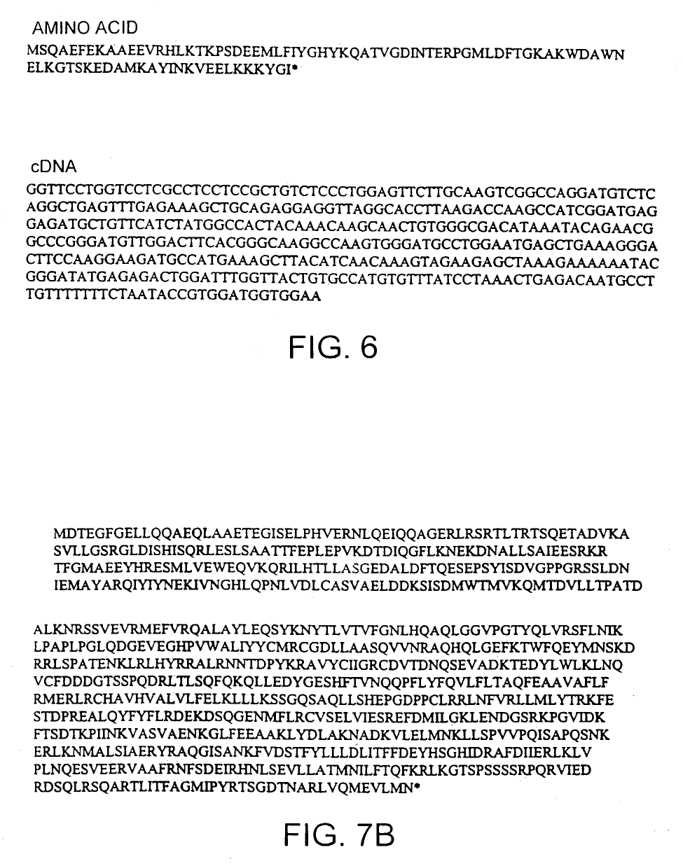

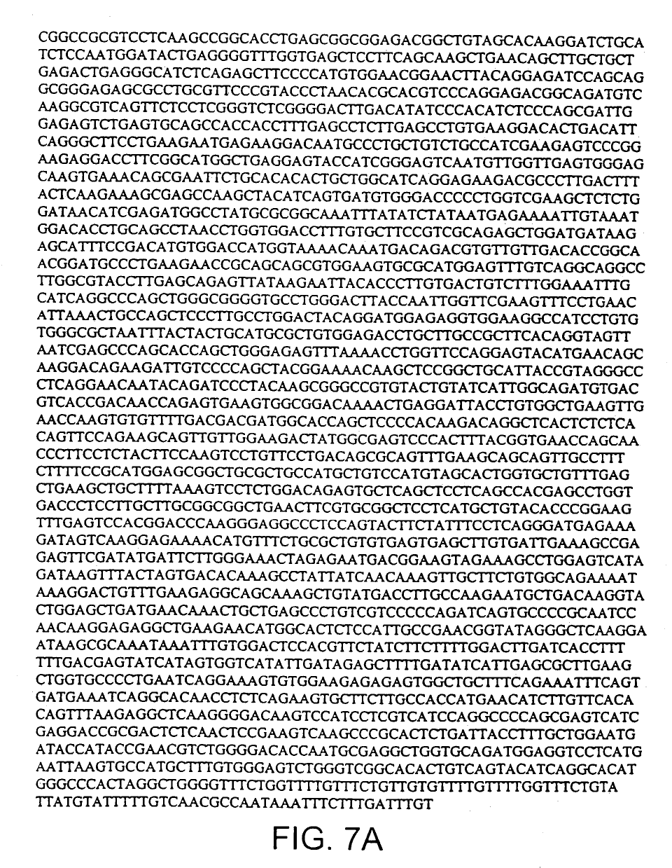

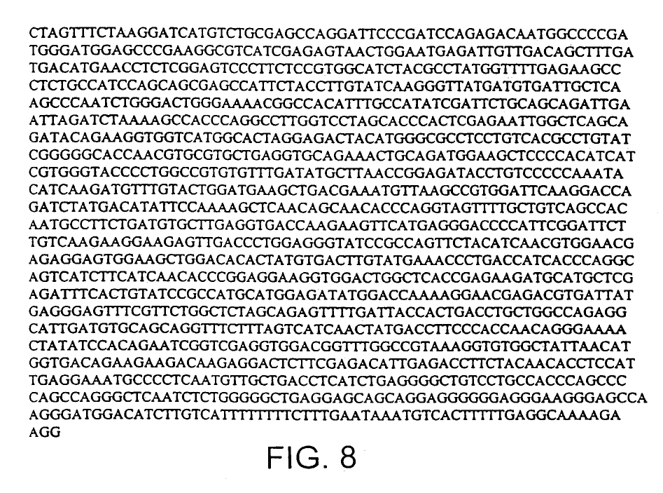

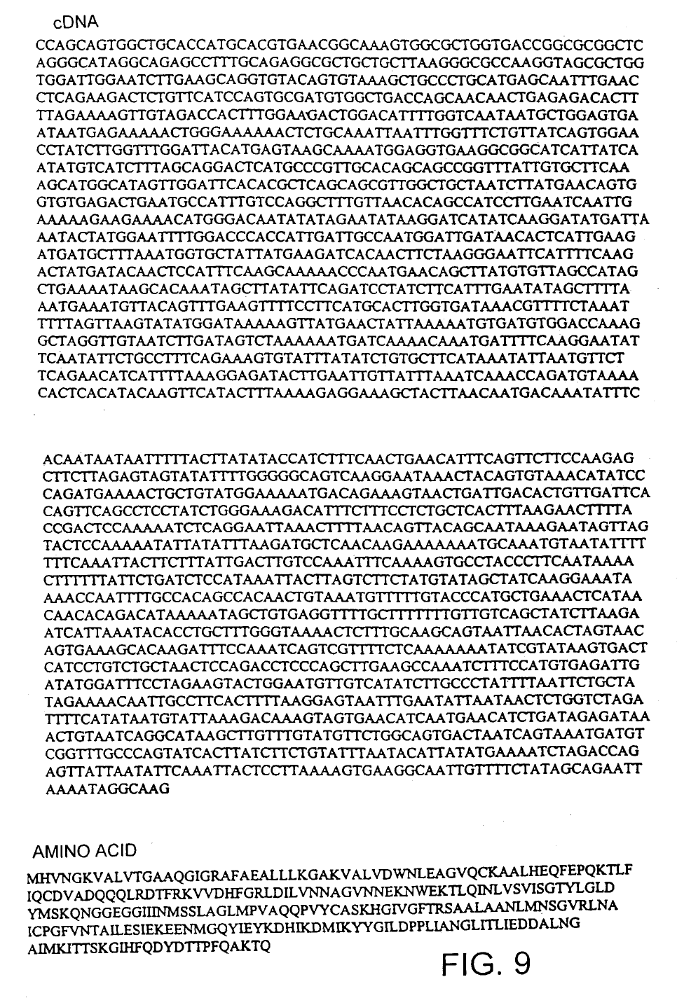

Among the clones selected for further analysis were HRPCa 2, HPRCa 3, HRPCa 6/7, HRPCa 8, HRPCa 9, HRPCa 10, HRPCa 13, HRPCa 14, HRPCa 15, and HRPCa 19. HRPCa 9 and HRPCa 10 appear to be novel genes. A full-length CDNA sequence for each gene was determined. These sequences, shown in FIGS. 1-9, as well as the known sequences of HRPCa 2, HPRCa 3, HRPCa 6/7, HRPCa 8, HRPCa 13, HRPCa 14, HRPCa 15, or HRPCa 19 can be used to produce the polypeptides and nucleic acids of the invention.

### Example 2

### Screening for Compounds Useful for the Treatment of Prostate Cancer

Compounds potentially useful for the treatment of prostate cancer can be identified as follows. Prostate cancer cells (e.g., WT LNCaP cells) are stably transfected with a vector capable of constitutively expressing HRPCa 2, HPRCa 3, HRPCa 6/7, HRPCa 8, HRPCa 9, HRPCa 10, HRPCa 13, HRPCa 14, HRPCa 15, or HRPCa 19(e.g., a vector in which expression of HRPCa 2, HPRCa 3, HRPCa 6/7, HRPCa 8, HRPCa 9, HRPCa 10, HRPCa 13, HRPCa 14, HRPCa.15, or HRPCa 19 is under the control of the CMV IE promoter). The transfected WT LNCaP cells are cultured under suitable conditions (e.g., in T162 flasks coated with Matrigel in RPMI-1640 medium supplemented with 10% FBS and 50 nM testosterone) in the presence and absence of a test compound and the growth rate of the cells is measured. A compound which reduces the growth rate of the cells is a potential therapeutic compound for the treatment of prostate cancer. A potential therapeutic compound identified in this manner can be further analyzed by determining its effect on the growth rate of androgen-independent prostate cancer cells (e.g., LN3 LNCaP cells).

The cDNA and protein sequences shown in the Figures and in publications or sequence databases referring to HRPCa 2, HPRCa 3, HRPCa 6/7, HRPCa 8, HRPCa 13, HRPCa 14, HRPCa 15, or HRPCa 19 provide those skilled in the art with the genes needed to prepared the transfected cell lines useful in for screening assays.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method-for identifying a compound useful for treating prostate cancer, comprising:
a) measuring the expression level of HRPCa 2, HPRCa 3, HRPCa 6/7, HRPCa 8, HRPCa 9, HRPCa 10, HRPCa 13, HRPCa 14, HRPCa 15, HRPCa 19, or peripheral-type benzodiazepine receptor (PBR) in a cell in the presence of a test compound; and
b) comparing the expression measured in step a) to the expression of HRPCa 2, HPRCa 3, HRPCa 6/7, HRPCa 8, HRPCa 9, HRPCa 10, HRPCa 13, HRPCa 14, HRPCa 15, HRPCa 19, or PBR in a cell in the absence of the compound,
wherein the compound is useful for treating prostate cancer when the expression level of HRPCa 2, HPRCa 3, HRPCa 6/7, HRPCa 8, HRPCa 9, HRPCa 10, HRPCa 13, HRPCa 14, HRPCa 15, HRPCa 19, or PBR in the presence of the test compound is less than its expression level in the absence of the test compound.

2. The method of claim 1 wherein the expression level is determined by measuring the amount of HRPCa 2, HPRCa 3, HRPCa 6/7, HRPCa.8, HRPCa 9, HRPCa 10, HRPCa 13, HRPCa 14, HRPCa 15, HRPCa 19, or PBR mRNA.

3. The method of claim 1 wherein the expression level is determined by measuring the amount of HRPCa 2, HPRCa 3, HRPCa 6/7, HRPCa 8, HRPCa 9, HRPCa 10, HRPCa 13, HRPCa 14, HRPCa 15, HRPCa 19, or PBR protein.

4. The method of claim 1 wherein the cell is a prostate cancer cell.

5. The method of claim 1 further comprising measuring the expression level of HRPCa 2, HPRCa 3, HRPCa 6/7, HRPCa 8, HRPCa 9, HRPCa 10, HRPCa 13, HRPCa 14, HRPCa 15, HRPCa 19, or PBR in the cell in the absence of the test compound.

6. The method of claim 4 wherein the prostate cancer cell is an androgen independent prostate cancer cell.

7. The method of claim 4 wherein the prostate cancer cell constitutively expresses HRPCa 2, HPRCa 3, HRPCa 6/7, HRPCa 8, HRPCa 9, HRPCa 10, HRPCa 13, HRPCa 14, HRPCa 15, HRPCa 19, or PBR.

8. A method for identifying a compound useful for treating prostate cancer, comprising
a) measuring an activity of HRPCa 2, HPRCa 3, HRPCa 6/7, HRPCa 8, HRPCa 9, HRPCa 10, HRPCa 13, HRPCa 14, HRPCa 15, HRPCa 19, or peripheral-type benzodiazepine receptor (PBR) in the presence of the compound; and
b) comparing the activity measured in step a) to the level of activity of HRPCa 2, HPRCa 3, HRPCa 6/7, HRPCa 8, HRPCa 9, HRPCa 10, HRPCa 13, HRPCa 14, HRPCa 15, HRPCa 19, or PBR in the absence of the test compound,
wherein the compound is useful for treating prostate cancer when the activity of HRPCa 2, HPRCa 3, HRPCa 6/7, HRPCa 8, HRPCa 9, HRPCa 10, HRPCa 13, HRPCa 14, HRPCa 15, HRPCa 19, or PBR in the presence of the test compound is lower than its activity in the absence of the test compound.

9. The method of claim 8 wherein the activity is measured in a cell.

10. The method of claim 9 wherein the cell is a prostate cancer cell.

11. The method of claim 10 wherein in the prostate cancer cell is an androgen-independent prostate cancer cell.

12. A method for identifying a compound useful for treating prostate cancer, comprising
a) measuring the growth of a sample of cells which constitutively express HRPCa 2, HPRCa 3, HRPCa 6/7, HRPCa 8, HRPCa 9, HRPCa 10, HRPCa 13, HRPCa 14, HRPCa 15, HRPCa 19, or peripheral-type benzodiazepine receptor (PBR) in the presence of the test compound; and
b) comparing the growth measured in step a) to the growth of a sample of the cells in the absence of the test compound,
wherein the compound is useful for treating prostate cancer when the growth of the sample of cells in the presence of the test compound is lower than the growth of the sample of cells in the absence of the test compound.

13. The method of claim 12 wherein the cell sample comprises prostate cancer cells.

14. The method of claim 13 wherein the prostate cancer cells are androgen-independent prostate cancer cells.

15. The method of claim 13 wherein growth is measured in the presence of an androgen.

16. A method of treating prostate cancer in a patient, comprising administering to the patient a compound which decreases the expression of HRPCa 2, HPRCa 3, HRPCa 6/7, HRPCa 8, HRPCa 9, HRPCa 10, HRPCa 13, HRPCa 14, HRPCa 15, HRPCa 19, or peripheral-type benzodiazepine receptor (PBR).

17. The method of claim 16 wherein the compound decreases expression of HRPCa 2, HPRCa 3, HRPCa 6/7, HRPCa 8, HRPCa 9, HRPCa 10, HRPCa 13, HRPCa 14, HRPCa 15, HRPCa 19, or PBR mRNA.

18. The method of claim 16 wherein the compound decreases expression of HRPCa 2, HPRCa 3, HRPCa 6/7, HRPCa 8, HRPCa 9, HRPCa 10, HRPCa 13, HRPCa 14, HRPCa 15, HRPCa 19, or PBR protein.

19. The method of claim 16 wherein the prostate cancer is androgen-dependent.

20. The method of claim 16 wherein the prostate cancer is androgen-independent.

21. A method of treating prostate cancer in a patient, comprising administering-to the patient a compound which decreases an activity of HRPCa 2,HPRCa 3, HRPCa 6/7, HRPCa 8, HRPCa 9, HRPCa 10, HRPCa 13, HRPCA 14, HRPCa 15, HRPCa 19, or peripheral benzodiazepine receptor (PBR).

22. The method of claim 21 wherein the prostate cancer is androgen-dependent.

23. The method of claim 22 wherein the prostate cancer is androgen-independent.

24. A method for diagnosing androgen-independent prostate cancer in a patient undergoing androgen withdrawal treatment, comprising:
a) providing a biological sample from the patient comprising prostate cells;
b) measuring the expression of HRPCa 2, HPRCa 3, HRPCa 6/7, HRPCa 8, HRPCa 9, HRPCa 10, HRPCa 13, HRPCa 14, HRPCa 15, HRPCa 19, or peripheral-type benzodiazepine receptor (PBR) in the sample in the presence and absence of androgen;
c) determining whether the expression of HRPCa 2, HPRCa 3, HRPCa 6/7, HRPCa 8, HRPCa 9, HRPCa 10, HRPCa 13, HRPCa 14, HRPCa 15, HRPCa 19, or PBR is increased in the presence androgen compared to the absence of androgen,
wherein the prostate cancer cells are androgen independent if the expression HRPCa 2, HPRCa 3, HRPCa 6/7, HRPCa 8, HRPCa 9, HRPCa 10, HRPCa 13, HRPCa 14, HRPCa 15, HRPCa 19, or PBR is not increased in the presence of androgen compared to the absence of androgen.

25. A method for determining the efficacy of androgen withdrawal treatment in a prostate cancer patient, comprising:
a) providing a first biological sample from the patient at a first time point and determining the expression level of HRPCa 2, HPRCa 3, HRPCa 6/7, HRPCa 8, HRPCa 9, HRPCa 10, HRPCa 13, HRPCa 14, HRPCa 15, HRPCa 19, or peripheral benzodiazepine receptor (PBR) in the first sample;
b) providing a second biological sample from the patient at a second time point after the first time point, the second time point occurring after the patient begins androgen withdrawal treatment, and determining the expression level of HRPCa 2, HPRCa 3, HRPCa 6/7, HRPCa 8, HRPCa 9, HRPCa 10, HRPCa 13, HRPCa 14, HRPCa 15, HRPCa 19, or PBR in the second sample;
c) comparing the expression level of HRPCa 2, HPRCa 3, HRPCa 6/7, HRPCa 8, HRPCa 9, HRPCa 10, HRPCa 13, HRPCa 14, HRPCa 15, HRPCa 19, or PBR in the first sample with that in the second sample, wherein an increase in the expression level in the second sample compared with the first sample indicates that the androgen withdrawal treatment has become less effective.

26. The method of claim 16 further comprising co-administering an anti-androgenic agent.

27. A method for determining whether a patient is suffering from androge-independent prostate cancer, comprising:
a) providing a sample of prostate cells from the patient;
a) measuring the expression of one or more of HRPCa 2, HPRCa 3, HRPCa 6/7, HRPCa 8, HRPCa 9, HRPCa 10, HRPCa 13, HRPCa 14, HRPCa 15, HRPCa 19, or peripheral benzodiazepine receptor (PBR) in the prostate cells in the presence and absence of an androgen;
b) determining that the patient is suffering from androgen independent prostate cancer when the expression of one or more of HRPCa 2, HPRCa 3, HRPCa 6/7, HRPCa 8, HRPCa 9, HRPCa 10, HRPCa 13, HRPCa 14, HRPCa 15, HRPCa 19, and PBR is not higher in the presence of the androgen than in the absence of the androgen.

28. A method for determining whether a patient suffering from prostate cancer should be treated with an agent which decreases the expression of HRPCa 2, HPRCa 3, HRPCa 6/7, HRPCa 8, HRPCa 9, HRPCa 10, HRPCa 13, HRPCa 14, HRPCa 15, HRPCa 19, or peripheral benzodiazepine receptor (PBR) comprising:
a) measuring the expression of one or more of HRPCa 2, HPRCa 3, HRPCa 6/7, HRPCa 8, HRPCa 9, HRPCa 10, HRPCa 13, HRPCa 14, HRPCa 15, HRPCa 19, and PBR in the prostate cells in the presence and absence of an androgen;
b) determining that the patient should be treated with an agent which decreases the expression of HRPCa 2, HPRCa 3, HRPCa 6/7, HRPCa 8, HRPCa 9, HRPCa 10, HRPCa 13, HRPCa 14, HRPCa 15, HRPCa 19, or PBR when the expression of one or more of HRPCa 2, HPRCa 3, HRPCa 6/7, HRPCa 8, HRPCa 9, HRPCa 10, HRPCa 13, HRPCa 14, HRPCa 15, HRPCa 19, and PBR is not higher in the presence of the androgen than in the absence of the androgen.


SEQUENCE LISTING (18 sequences)

SEQ ID NO: 1 - DNA, 1429 bp, Homo sapiens
  CDS (82)...(528)
ggcggctcggactgagcaggactttccttatcccagttgattgtgcagaatacactgcctgtcgcttgtcttctattcaccatggcttcttctgatatccaggtgaaagaactggagaagcgtgcctcaggccaggcttttgagctgattctcagccctcggtcaaaagaatctgttccagaattccccctttcccctccaaagaagaaggatctttccctggaggaaattcagaagaaattagaagctgcagaagaaagacgcaagtcccatgaagctgaggtcttgaagcagctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaaggataagcacattgaagaagtgcggaagaacaaagaatccaaagaccctgctgacgagactgaagctgactaatttgttctgagaactgactttctccccatccccttcctaaatatccaaagactgtactggccagtgtcattttattttttccctcctgacaaatattttagaagctaatgtaggactgtataggtagatccagatccagactgtaagatgttgttttaggggctaaaggggagaaactgaaagtgttttactctttttctaaagtgttggtctttctaatgtagctatttttcttgttgcatcttttctacttcagtacacttggtgtactgggttaatggctagtactgtattggctctgtgaaaacatatttgtgaaaagagtatgtagtggcttcttttgaactgttagatgctgaatatctgttcacttttcaatcccaattctgtcccaatcttaccagatgctactggacttgaatggttaataaaactgcacagtgctgttggtggcagtgacttcttttgagttaggttaataaatcaagccatagagcccctcctggttgatacttgttccagatggggcctttggggctggtagaaatacccaacgcacaaatgaccgcacgttctctgccccgtttcttgccccagtgtggtttgcattgtctccttccacaatgactgctttgtttggatgcctcagcccaggtcagctgttactttctttcagatgtttatttgcaaacaaccattttttgttctgtgtcccttttaaaaggcagattaaaagcacaagcgtgtttctagagaacagttgagagagaatctcaagattctacttggtggtttgcttgctctacgttacaggtggggcatgtcctcatcctttcctgccataaaagctatgacacgagaatcagaatattaataaaactttatgtactgctgtagt

SEQ ID NO: 2 - protein, 149 aa, Homo sapiens
MASSDIQVKELEKRASGQAFELILSPRSKESVPEFPLSPPKKKDLSLEEIQKKLEAAEERRKSHEAEVLKQLAEKREHEKEVLQKAIEENNNFSKMAEEKLTHKMEANKENREAQMAAKLERLREKDKHIEEVRKNKESKDPADETEAD

SEQ ID NO: 3 - DNA, 1188 bp, Homo sapiens
  CDS (109)...(714)
tcgagatccattgtgctctaaaggctcgccctcctgtgcatcgcggctaatttggggtatcactgagctgaagacaaagagaagggggagaaaacctagcagaccaccatgtgctatgggaagtgtgcacgatgcatcggacattctctggtggggctcgccctcctgtgcatcgcggctaatattttgctttactttcccaatggggaaacaaagtatgcctccgaaaaccacctcagccgcttcgtgtggttcttttctggcatcgtaggaggtggcctgctgatgctcctgccagcatttgtcttcattgggctggaacaggatgactgctgtggctgctgtggccatgaaaactgtggcaaacgatgtgcgatgctttcttctgtattggctgctctcattggaattgcaggatctggctactgtgtcattgtggcagcccttggcttagcagaaggaccactatgtcttgattccctcggccagtggaactacacctttgccagcaccgagggccagtaccttctggatacctccacatggtccgagtgcactgaacccaagcacattgtggaatggaatgtatctctgttttctatcctcttggctcttggtggaattgaattcatcttgtgtcttattcaagtaataaatggagtgcttggaggcatatgtggcttttgctgctctcaccaacagcaatatgactgctaaaagaaccaacccaggacagagccacaatcttcctctatttcattgtaatttatatatttcacttgtattcatttgtaaaactttgtattagtgtaacatactccccacagtctacttttacaaacgcctgtaaagactggcatcttcacaggatgtcagtgtttaaatttagtaaacttcttttttgtttgtttatttgtgtaacatactccccacagtctacttttacaaacgcctgtaaagactggcatcttcacaggatgtcagtgtttaaatttagtaaacttcttttttgtttgtttatttgtttttgtttttttttaaggaatgaggaaacaaaccaccctctgggggtagtttacagactgagtgacagtactcagtatatctgagataaactctataatgttttggataaaaataacattccatggcacatatatacaatagtgattggctttagagcacaat

SEQ ID NO: 4 - protein, 202 aa, Homo sapiens
MCYGKCARCIGHSLVGLALLCIAANILLYFPNGETKYASENHLSRFVWFFSGIVGGGLLMLLPAFVFIGLEQDDCCGCCGHENCGKRCAMLSSVLAALIGIAGSGYCVIVAALGLAEGPLCLDSLGQWNYTFASTEGQYLLDTSTWSECTEPKHIVEWNVSLFSILLALGGIEFILCLIQVINGVLGGICGFCCSHQQQYDC

SEQ ID NO: 5 - DNA, 3244 bp, Homo sapiens
  CDS (145)...(1620)
gtcgacccacgcgtccgcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggatcaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtttagcctgcggggtcaacttgaactcaagccgccagagcaggatcgtgggcggtgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtacaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggttggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcttgcaatcccatttgcaggatccgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcgatttttttttcaaagtaaaaaaaaaaaaaaaaaaaaaaagggcggccgc

SEQ ID NO: 6 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGSNFILQVYSSQRKSWHPVCQDDWNENYGRAACRDMGYKNNFYSSQGIVDDSGSTSFMKLNTSAGNVDIYKKLYHSDACSSKAVVSLRCLACGVNLNSSRQSRIVGGESALPGAWPWQVSLHVQNVHVCGGSIITPEWIVTAAHCVEKPLNNPWHWTAFAGILRQSFMFYGAGYQVQKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 7 - DNA, 903 bp, Homo sapiens
  CDS (231)...(611)
ctgcaggtaccggtccggaattcccgggtcgacccacgcgtccgccttcgcggggcgcggkccgcagcttttcgggtcgacccacgcgtccggccgcgggaagggtactccaggcgagaggcggacgcgagtcgtcgtggcaggaaaagtgactagctccccttcgttgtcagccagggacgagaacacagccacgctcccacccggctgccaacgatccctcggcggcgatgtcggccgccggtgcccgaggcctgcgggccacctaccaccggctcctcgataaagtggagctgatgctgcccgagaaattgaggccgttgtacaaccatccagcaggtcccagaacagttttcttctgggctccaattatgaaatgggggttggtgtgtgctggattggctgatatggccagacctgcagaaaaacttagcacagctcaatctgctgttttgatggctacagggtttatttggtcaagatactcacttgtaattattccaaaaaattggagtctgtttgctgttaatttctttgtgggggcagcaggagcctctcagctttttcgtatttggagatataaccaagaactaaaagctaaagcacacaaataaaagagttcctgatcacctgaacaatctagatgtggacaaaaccattgggacctagtttattatttggttattgataaagcaaagctaactgtgtgtttagaaggcactgtaactggtagctagttcttgattcaatagaaaaatgcagcaaacttttaataacagtctctctacatgacttaaggaacttatctatggatattagtaacatttttctaccatttgtccgtaataaaccatacttgctcgtaaaaaaaaaaaaaaaaaagggcggccgcaaagggcggcc

SEQ ID NO: 8 - protein, 127 aa, Homo sapiens [sequence omitted]

SEQ ID NO: 9 - DNA, 4697 bp, Homo sapiens
  CDS (102)...(1601)
gtcgacccacgcgtccggcgtagtgcgctccccgcccagcctgcagagctcgcgccgcggcagcccagccgctcggccccgccgcgctcgcagaggccgccatgggcaccgcgcgctggctcgcgctgggcagcctcttcgccctggctgggctgctggaaggccggctcgtgggcgaggaggaagccggctttggcgaatgtgacaagttcttctacgccgggaccccgcctgcggggctggcggccgattcccacgtgaagatctgtcagcgcgcggagggtgctgagcgcttcgccaccctctacagcacccgggaccgcatccccgtgtactccgcgttccgcgccccgcgccctgcgcccggcggcgccgagcagcgatggctggtggagccgcagatcgatgaccccaacagcaaccttgaggaggcgattaatgaggcagaggccatcacctctgtgaacagcctgggaagcaagcaagccttgaatacagattaccttgattctgattaccaaagaggacagctttacccattctcccttagcagtgatgtccaggtggccacatttactctcacaaattcagccccaatgactcagtccttccaggaacggtggtatgtgaatctccacagcctaatggaccgggctttgaccccacagtgtggcagtggggaagacctatatatcctcacaggcacagtgccctcagactacagagttaaagacaaagtggcagtccctgagtttgtttggctggcagcctgttgtgctgtccctggaggaggctgggccatgggctttgtcaagcacacccgggacagtgacatcatagaagatgtgatggtaaaagatcttcagaaactgcttccatttaaccctcagctgtttcagaacaactgtggtgaaactgagcaagacacagagaaaatgaaaaaaatcctggaagtggttaaccaaatccaggatgaagaacgaatggtacaatctcaaaagagttctagtcccctttctagcaccaggagcaagaggtctactctgttgcctccagaggcatctgagggaagtagtagctttttgggaaaactcatgggcttcattgctaccccattcatcaagctttttcaattaatttattaccttgtggtagcaatcctgaagaacattgtctatttcctgtggtgtgttaccaagcaggtgattaatggcatagaaagttgcctttaccgcctgggctcagccaccatctcatacttcatggccattggggaagagttggtgagcattccctggaaggtgctcaaggtcgtggccaaagtcatcagggctctcctccggatcctttgttgtctgctgaaggccatttgccgagttctgagcatccctgtccgtgtccttgtggatgtggccactttccctgtgtacaccatggtcgctattccaattgtttgcaaggacattgcactgggccttggtggcactgtctcactgctctttgacactgcttttggtaccctgggtggcctatttcaggtggtttttagtgtctgcaagcggattggctacaaggttacttttgacaattctggggagttataaactcaaaaaactaatagtatccagtcacagtgaatttgaaagctggaatagtttgtctttacaatgggtttctgttcactgtcagttatcattatattttggcctttggtggggatgtctgcttgtttttgcaaaagaagatggcagaatttagacttgacagaggagaaatgctcagggtgagattaggtgtagtaatctgctgtttacctccagttatatgtgcaaactcccaagccactaataacttcagttatgcactctaacacagacgaccacctgaaatgcactggtatttatttctgataattaaaaattacaggggagggaagaactagaaaaagaacaactttagaccaaaggtgtctgagaaaaggagaaagggagcttgttcttcccattgctctttgtgatttaaggcaaaacagattaaaaaaaaaatctgcagccaatttcttggcatttgcttctctttctcctcaactcgactgaccttggtggaatgcagataatgcctctttgttgaaataactttatgggaatgtacaattttctatatctcttagctttccgtggttctcaaggatatgtacagttttcattttcctccaaagttgaattttgctacatttttcttttaggtaatgataagcattttttaaaaaatcatttttaggtaatggtaagcattttatgccaaatgtggcataacagagtttgaattgaagggcaaagttttcttttcttttttttttggccccttgaatggtataatacagtcctctccggtggaaagaagagaagagaaggtggacagccctgctcttagtaggtgctgcagatccaaggacatcttttgtccagcttggattaacttgacgtgtatccctgcctgacaaggttgaactgaaagatctatatgttaagctaacatgaaaattcatattctgcaacatagtagatttttctaatgcatgaaataagtacccagcacagtaaaaatactctgacttatgtccctaaatggttgttttgatacaactatatagaaaagagccacaaaataaagataaaagttattgtggccatctctgaaaaaaatatataaaatatttaagaataattatatcttaggaaattatttttacagtgtgtttgaggctacaaacataactcccccattaatacaaattaaatgtgagagctcattctcaaaatttttttgatcaagcacttgtcattttaaatcttgcactaaaaaatggtaacaagagggaccaaactttgcttcccacaattgggatggaatcacctggatttttctgaatgttttaaagaattgctgagaggtagaaacagccaagtatgaaatactgatcttggggctaccgcccaggatcaatcagaaagttatatgcaaaaattcggggtcccaacaaggagagaacaatatgtcaaccatttgcatggggtcgatggatgagaaacatgagcgtagcaagagttacattttgcagaaaaatagcggaggcacacccagagtagaacagcagccagcaagctgccatcctgatcaatttgtgatgcaaggttagggagtatgagcaccgcatgggtccatgctagggagatgtgcaccaggcttagctaagaaacttaaagcagtattttaccaactcttgtcttagggagcataaagtttggatgtccctttatttcagcagtgtgaaggtaaatggaagggtgagggtttgacttggtactgattggtcaagaatcctgctggataaagaaaaggaattttagaagtgaccagagggacagtccagccaaactattatttgataaggaacccaaggccctgggaggcaacaggctagcccttagtttcatggctagctgagagcagattaggagccaacgttgtttgcacatgtcccatcacacctgagatgtcagacatgggaagttcgtgctattattcagttgcctctctggaccatggcaagatttcctcattcatcaaacagactccaggccctgacaaagcagttggatttggcatgtgtgatgaaaacaattagccatccatgtacaacattatgcttactgcatcccatggaaacttaattccctcctagcacatatttgcatactgaaaggtccgaaaagggcatccacggcagcttgagccccttagcaccatgtaaggagcacagcatccaaacggcttcttgagaaccattggggaatgtctttctttttcacatccaattgtttagtgtctatttattcttgggtggccagttttgaaacctaaaaagggacaataaagcaaaaagtatcagtaaggataggtggctgagacccactgccctgagctactagtgtggctgtgcctgtgggtctctagaaccatctgcattggacgtgaagccacagcaggtggctggactgctggcctgttcctaatgagctacgctgggctttgaggtagaggttggggtttatgaaccccaactctggcttaaagatcttgctgtggctctgttatgttctgaggccttgggattagcctcttcctcattatggagctgattttctagtctgtggatcagctatgcctttggacacttctcttttccattgtgccttttgaatgttgtcttctcactcagcatcagcacttcgatctaaatgcagactaggtagttgggaggaggaaccaaagtgaaccatccttcatttattcagtcattcgttcatctgtcaaacacgtatttggacatcaaggttgcagagatgaacaatgcatggatttcatctttgaggagttcaaaacctagtggagagaacacatggtacaatcgtaacacatgaaggacaagtaagtgctgcagtaaaggtactaataacatgttccttggaacagaggaagaaaaaccacgaaaccatggaaattagggaagcctttacagagggtgtgacaaaactcaatttgacattttcaagctatgtacaatgatgtgcaccttgcagatgctcaataaagtaattactgacaaaaaaaaaaaaaaaaaaaagggcggccgc

SEQ ID NO: 10 - protein, 500 aa, Homo sapiens [sequence omitted]

SEQ ID NO: 11 - DNA, 405 bp, Homo sapiens
  CDS (58)...(318)
ggttcctggtcctcgcctcctccgctgtctccctggagttcttgcaagtcggccaggatgtctcaggctgagtttgagaaagctgcagaggaggttaggcaccttaagaccaagccatcggatgaggagatgctgttcatctatggccactacaaacaagcaactgtgggcgacataaatacagaacggcccgggatgttggacttcacgggcaaggccaagtgggatgcctggaatgagctgaaagggacttccaaggaagatgccatgaaagcttacatcaacaaagtagaagagctaaagaaaaaatacgggatatgagagactggatttggttactgtgccatgtgtttatcctaaactgagacaatgccttgtttttttctaataccgtggatggtggaa

SEQ ID NO: 12 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

SEQ ID NO: 13 - DNA, 2681 bp, Homo sapiens
  CDS (67)...(2523)
cggccgcgtcctcaagccggcacctgagcggcggagacggctgtagcacaaggatctgcatctccaatggatactgaggggtttggtgagctccttcagcaagctgaacagcttgctgctgagactgagggcatctcagagcttccccatgtggaacggaacttacaggagatccagcaggcgggagagcgcctgcgttcccgtaccctaacacgcacgtcccaggagacggcagatgtcaaggcgtcagttctcctcgggtctcggggacttgacatatcccacatctcccagcgattggagagtctgagtgcagccaccacctttgagcctcttgagcctgtgaaggacactgacattcagggcttcctgaagaatgagaaggacaatgccctgctgtctgccatcgaagagtcccggaagaggaccttcggcatggctgaggagtaccatcgggagtcaatgttggttgagtgggagcaagtgaaacagcgaattctgcacacactgctggcatcaggagaagacgcccttgactttactcaagaaagcgagccaagctacatcagtgatgtgggaccccctggtcgaagctctctggataacatcgagatggcctatgcgcggcaaatttatatctataatgagaaaattgtaaatggacacctgcagcctaacctggtggacctttgtgcttccgtcgcagagctggatgataagagcatttccgacatgtggaccatggtaaaacaaatgacagacgtgttgttgacaccggcaacggatgccctgaagaaccgcagcagcgtggaagtgcgcatggagtttgtcaggcaggccttggcgtaccttgagcagagttataagaattacacccttgtgactgtctttggaaatttgcatcaggcccagctgggcggggtgcctgggacttaccaattggttcgaagtttcctgaacattaaactgccagctcccttgcctggactacaggatggagaggtggaaggccatcctgtgtgggcgctaatttactactgcatgcgctgtggagacctgcttgccgcttcacaggtagttaatcgagcccagcaccagctgggagagtttaaaacctggttccaggagtacatgaacagcaaggacagaagattgtccccagctacggaaaacaagctccggctgcattaccgtagggccctcaggaacaatacagatccctacaagcgggccgtgtactgtatcattggcagatgtgacgtcaccgacaaccagagtgaagtggcggacaaaactgaggattacctgtggctgaagttgaaccaagtgtgttttgacgacgatggcaccagctccccacaagacaggctcactctctcacagttccagaagcagttgttggaagactatggcgagtcccactttacggtgaaccagcaacccttcctctacttccaagtcctgttcctgacagcgcagtttgaagcagcagttgcctttcttttccgcatggagcggctgcgctgccatgctgtccatgtagcactggtgctgtttgagctgaagctgcttttaaagtcctctggacagagtgctcagctcctcagccacgagcctggtgaccctccttgcttgcggcggctgaacttcgtgcggctcctcatgctgtacacccggaagtttgagtccacggacccaagggaggccctccagtacttctatttcctcagggatgagaaagatagtcaaggagaaaacatgtttctgcgctgtgtgagtgagcttgtgattgaaagccgagagttcgatatgattcttgggaaactagagaatgacggaagtagaaagcctggagtcatagataagtttactagtgacacaaagcctattatcaacaaagttgcttctgtggcagaaaataaaggactgtttgaagaggcagcaaagctgtatgaccttgccaagaatgctgacaaggtactggagctgatgaacaaactgctgagccctgtcgtcccccagatcagtgccccgcaatccaacaaggagaggctgaagaacatggcactctccattgccgaacggtatagggctcaaggaataagcgcaaataaatttgtggactccacgttctatcttcttttggacttgatcaccttttttgacgagtatcatagtggtcatattgatagagcttttgatatcattgagcgcttgaagctggtgcccctgaatcaggaaagtgtggaagagagagtggctgctttcagaaatttcagtgatgaaatcaggcacaacctctcagaagtgcttcttgccaccatgaacatcttgttcacacagtttaagaggctcaaggggacaagtccatcctcgtcatccaggccccagcgagtcatcgaggaccgcgactctcaactccgaagtcaagcccgcactctgattacctttgctggaatgataccataccgaacgtctggggacaccaatgcgaggctggtgcagatggaggtcctcatgaattaagtgccatgctttgtgggagtctgggtcggcacactgtcagtacatcaggcacatgggcccactaggctggggtttctggttttgtttctgttgtgttttgttttggtttctgtattatgtatttttgtcaacgccaataaatttctttgatttgt

SEQ ID NO: 14 - protein, 819 aa, Homo sapiens
MDTEGFGELLQQAEQLAAETEGISELPHVERNLQEIQQAGERLRSRTLTRTSQETADVKASVLLGSRGLDISHISQRLESLSAATTFEPLEPVKDTDIQGFLKNEKDNALLSAIEESRKRTFGMAEEYHRESMLVEWEQVKQRILHTLLASGEDALDFTQESEPSYISDVGPPGRSSLDNIEMAYARQIYIYNEKIVNGHLQPNLVDLCASVAELDDKSISDMWTMVKQMTDVLLTPATDALKNRSSVEVRMEFVRQALAYLEQSYKNYTLVTVFGNLHQAQLGGVPGTYQLVRSFLNIKLPAPLPGLQDGEVEGHPVWALIYYCMRCGDLLAASQVVNRAQHQLGEFKTWFQEYMNSKDRRLSPATENKLRLHYRRALRNNTDPYKRAVYCIIGRCDVTDNQSEVADKTEDYLWLKLNQVCFDDDGTSSPQDRLTLSQFQKQLLEDYGESHFTVNQQPFLYFQVLFLTAQFEAAVAFLFRMERLRCHAVHVALVLFELKLLLKSSGQSAQLLSHEPGDPPCLRRLNFVRLLMLYTRKFESTDPREALQYFYFLRDEKDSQGENMFLRCVSELVIESREFDMILGKLENDGSRKPGVIDKFTSDTKPIINKVASVAENKGLFEEAAKLYDLAKNADKVLELMNKLLSPVVPQISAPQSNKERLKNMALSIAERYRAQGISANKFVDSTFYLLLDLITFFDEYHSGHIDRAFDIIERLKLVPLNQESVEERVAAFRNFSDEIRHNLSEVLLATMNILFTQFKRLKGTSPSSSSRPQRVIEDRDSQLRSQARTLITFAGMIPYRTSGDTNARLVQMEVLMN

SEQ ID NO: 15 - DNA, 1383 bp, Homo sapiens
  CDS (17)...(1234)
ctagtttctaaggatcatgtctgcgagccaggattcccgatccagagacaatggccccgatgggatggagcccgaaggcgtcatcgagagtaactggaatgagattgttgacagctttgatgacatgaacctctcggagtcccttctccgtggcatctacgcctatggttttgagaagccctctgccatccagcagcgagccattctaccttgtatcaagggttatgatgtgattgctcaagcccaatctgggactgggaaaacggccacatttgccatatcgattctgcagcagattgaattagatctaaaagccacccaggccttggtcctagcacccactcgagaattggctcagcagatacagaaggtggtcatggcactaggagactacatgggcgcctcctgtcacgcctgtatcgggggcaccaacgtgcgtgctgaggtgcagaaactgcagatggaagctccccacatcatcgtgggtacccctggccgtgtgtttgatatgcttaaccggagatacctgtcccccaaatacatcaagatgtttgtactggatgaagctgacgaaatgttaagccgtggattcaaggaccagatctatgacatattccaaaagctcaacagcaacacccaggtagttttgctgtcagccacaatgccttctgatgtgcttgaggtgaccaagaagttcatgagggaccccattcggattcttgtcaagaaggaagagttgaccctggagggtatccgccagttctacatcaacgtggaacgagaggagtggaagctggacacactatgtgacttgtatgaaaccctgaccatcacccaggcagtcatcttcatcaacacccggaggaaggtggactggctcaccgagaagatgcatgctcgagatttcactgtatccgccatgcatggagatatggaccaaaaggaacgagacgtgattatgagggagtttcgttctggctctagcagagttttgattaccactgacctgctggccagaggcattgatgtgcagcaggtttctttagtcatcaactatgaccttcccaccaacagggaaaactatatccacagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatctgaggggctgtcctgccacccagccccagccagggctcaatctctgggggctgaggagcagcaggaggggggagggaagggagccaagggatggacatcttgtcattttttttctttgaataaatgtcactttttgaggcaaaagaagg

SEQ ID NO: 16 - protein, 406 aa, Homo sapiens
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI

SEQ ID NO: 17 - DNA, 2412 bp, Homo sapiens
  CDS (18)...(815)
ccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctgcatgagcaatttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttaatacattatatgaaaatctagaccagagttattaatattcaaattactccttaaaagtgaaggcaattgttttctatagcagaattaaaataggcaag

SEQ ID NO: 18 - protein, 266 aa, Homo sapiens
MHVNGKVALVTGAAQGIGRAFAEALLLKGAKVALVDWNLEAGVQCKAALHEQFEPQKTLFIQCDVADQQQLRDTFRKVVDHFGRLDILVNNAGVNNEKNWEKTLQINLVSVISGTYLGLDYMSKQNGGEGGIIINMSSLAGLMPVAQQPVYCASKHGIVGFTRSAALAANLMNSGVRLNAICPGFVNTAILESIEKEENMGQYIEYKDHIKDMIKYYGILDPPLIANGLITLIEDDALNGAIMKITTSKGIHFQDYDTTPFQAKTQ